(19) United States
(12) Patent Application Publication
(10) Pub. No.: US 2009/0035366 A1
(43) Pub. Date: Feb. 5, 2009

(54) Nanoparticulate benzothiophene formulations

(75) Inventors: Gary Liversidge, West Chester, PA (US); Scott Jenkins, Downingtown, PA (US)

Correspondence Address: Elan Drug Delivery, Inc. c/o Foley & Lardner, 3000 K Street, N.W., Suite 500, Washington, DC, 20007-5109, US

(73) Assignee: Elan Pharma International Ltd.

(21) Appl. No.: 12/232,895

(22) Filed: Sep. 25, 2008

Related U.S. Application Data
(63) Continuation of application No. 11/292,314, filed on Dec. 2, 2005.
(60) Provisional application No. 60/633,003, filed on Dec. 3, 2004.

(51) Int. Cl. A61K 9/48; A61K 31/4535; A61K 9/14; A61P 19/00; A61K 9/20; A61K 9/00

(52) U.S. Cl. 424/451; 514/324; 424/489; 424/484; 424/464

(57) ABSTRACT

The present invention is directed to benzothiophene compositions, preferably nanoparticulate raloxifene hydrochloride compositions having improved pharmacokinetic profiles, improved bioavailability, dissolution rates and efficacy. In one embodiment, the raloxifene hydrochloride nanoparticulate composition have an effective average particle size of less than about 2000 nm.

### CROSS-REFERENCE TO RELATED APPLICATIONS

This application is a continuation of U.S. application Ser. No. 11/292,314, filed Dec. 2, 2005, which claims priority from U.S. Provisional Patent Application No. 60/633,003, filed Dec. 3, 2004. The contents of these applications are incorporated by reference in their entirety.

### FIELD OF THE INVENTION

This invention relates to the fields of pharmaceutical and organic chemistry and provides a benzothiophene compound, such as a raloxifene hydrochloride compound, in nanoparticulate form, which is useful for the treatment of various medical indications, including osteoporosis.

### BACKGROUND OF THE INVENTION

Background Regarding Nanoparticulate Compositions

Osteoporosis describes a group of diseases which arise from diverse etiologies, but which are characterized by the net loss of bone mass per unit volume. The consequence of this loss of bone mass and resulting bone fracture is the failure of the skeleton to provide adequate structural support for the body. One of the most common types of osteoporosis is that associated with menopause. Most women lose from about 20% to about 60% of the bone mass in the trabecular compartment of the bone within 3 to 6 years after the cessation of menses. This rapid loss is generally associated with an increase of bone resorption and formation. However, the resorptive cycle is more dominant and the result is a net loss of bone mass. Osteoporosis is a common and serious disease among post-menopausal women.

There are an estimated 25 million women in the United States, alone, who are afflicted with this disease. The results of osteoporosis are personally harmful and also account for a large economic loss due to its chronicity and the need for extensive and long term support (hospitalization and nursing home care) from the disease sequelae. This is especially true in more elderly patients. Additionally, although osteoporosis is not generally thought of as a life threatening condition, a 20% to 30% mortality rate is related with hip fractures in elderly women. A large percentage of this mortality rate can be directly associated with post-menopausal osteoporosis.

Before menopause time, most women have less incidence of cardiovascular disease than age-matched men. Following menopause, however, the rate of cardiovascular disease in women slowly increases to match the rate seen in men. This loss of protection has been linked to the loss of estrogen and, in particular, to the loss of estrogen's ability to regulated the levels of serum lipids. The nature of estrogen's ability to regulate serum lipids is not well understood, but evidence to date indicates that estrogen can up regulate the low density lipid (LDL) receptors in the liver to remove excess cholesterol. Additionally, estrogen appears to have some effect on the biosynthesis of cholesterol, and other beneficial effects on cardiovascular health.

It has been reported in the literature that post-menopausal women having estrogen replacement therapy have a return of serum lipid levels to concentrations to those of the pre-menopausal state. Thus, estrogen would appear to be a reasonable treatment for this condition. However, the side-effects of estrogen replacement therapy are not acceptable to many women, thus limiting the use of this therapy. An ideal therapy for this condition would be an agent which would regulate the serum lipid level as does estrogen, but would be devoid of the side-effects and risks associated with estrogen therapy.

Preclinical findings with a structurally distinct “anti-estrogen”, raloxifene hydrochloride, have demonstrated potential for improved selectivity of estrogenic effects in target tissues. Similar to tamoxifen, raloxifene hydrochloride was developed originally for treatment of breast cancer; however, the benzothiophene nucleus of raloxifene hydrochloride represented a significant structural deviation from the triphenylethylene nucleus of tamoxifen. Raloxifene hydrochloride binds with high affinity to the estrogen receptor, and inhibits estrogen-dependent proliferation in MCF-7 cells (human mammary tumor derived cell line) in cell culture. In vivo estrogen antagonist activity of raloxifene hydrochloride was furthermore demonstrated in carcinogen-induced models of mammary tumors in rodents. Significantly, in uterine tissue raloxifene hydrochloride was more effective than tamoxifen as an antagonist of the uterotrophic response to estrogen in immature rats and, in contrast to tamoxifen, raloxifene hydrochloride displayed only minimal uterotrophic response that was not dose-dependent in ovariectomized (OVX) rats. Thus, raloxifene hydrochloride is unique as an antagonist of the uterine estrogen receptor, in that it produces a nearly complete blockage of uterotrophic response of estrogen due to minimal agonist effect of raloxifene hydrochloride in this tissue. Indeed, the ability of raloxifene hydrochloride to antagonize the uterine stimulatory effect of tamoxifen was recently demonstrated in OVX rats. Raloxifene hydrochloride is more properly characterized as a Selective Estrogen Receptor Modulator (SERM), due to its unique profile. The chemical structure of raloxifene hydrochloride is:

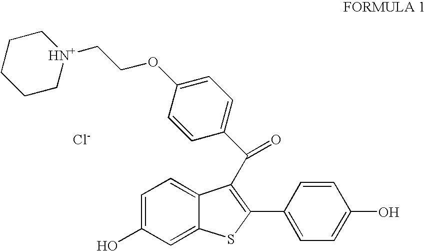

The chemical designation is methanone, [6-hydroxy-2-(4-hydroxyphenyl)benzo[b]thien-3-yl]-[4-[2-(1-piperidinyl)ethoxy]phenyl]-, hydrochloride. Raloxifene hydrochloride (HCl) has the empirical formula C28H27NO 4 S.HCl, which corresponds to a molecular weight of 510.05. Raloxifene HCl is an off-white to pale-yellow solid that is very slightly soluble in water.

Raloxifene HCL is commercially available in tablet dosage form for oral administration (Eli Lilly, Indianapolis, Ind.). Each tablet is the molar equivalent of 55.71 mg free base with inactive ingredients that include anhydrous lactose, carnuba wax, crospovidone, FD&C Blue #2, aluminum lake, hypromellose, lactose monohydrate, and magnesium stearate, as well as other commercially available excipients well know to the art.

Raloxifene hydrochloride and processes for its preparation are described and claimed in U.S. Pat. Nos. 5,393,763 and 5,457,117 to Black et al; 5,478,847 to Draper; 5,812,120 and 5,972,383 to Gibson et al., and 6,458,811 and 6,797,719 to Arbuthnat et al., all of which are incorporated herein by reference.

Nanoparticulate compositions, first described in U.S. Pat. No. 5,145,684 (“the '684 patent”), are particles consisting of a poorly soluble therapeutic or diagnostic agent having adsorbed onto, or associated with, the surface thereof a non-crosslinked surface stabilizer. The '684 patent does not describe nanoparticulate compositions of a benzothiophene.

Methods of making nanoparticulate compositions are described in, for example, U.S. Pat. Nos. 5,518,187 and 5,862,999, both for “Method of Grinding Pharmaceutical Substances;” U.S. Pat. No. 5,718,388, for “Continuous Method of Grinding Pharmaceutical Substances;” and U.S. Pat. No. 5,510,118 for “Process of Preparing Therapeutic Compositions Containing Nanoparticles.”

Nanoparticulate compositions are also described, for example, in U.S. Pat. Nos. 5,298,262 for “Use of Ionic Cloud Point Modifiers to Prevent Particle Aggregation During Sterilization;” 5,302,401 for “Method to Reduce Particle Size Growth During Lyophilization;” 5,318,767 for “X-Ray Contrast Compositions Useful in Medical Imaging;” 5,326,552 for “Novel Formulation For Nanoparticulate X-Ray Blood Pool Contrast Agents Using High Molecular Weight Non-ionic Surfactants;” 5,328,404 for “Method of X-Ray Imaging Using Iodinated Aromatic Propanedioates;” 5,336,507 for “Use of Charged Phospholipids to Reduce Nanoparticle Aggregation;” 5,340,564 for “Formulations Comprising Olin 10-G to Prevent Particle Aggregation and Increase Stability;” 5,346,702 for “Use of Non-Ionic Cloud Point Modifiers to Minimize Nanoparticulate Aggregation During Sterilization;” 5,349,957 for “Preparation and Magnetic Properties of Very Small Magnetic-Dextran Particles;” 5,352,459 for “Use of Purified Surface Modifiers to Prevent Particle Aggregation During Sterilization;” 5,399,363 and 5,494,683, both for “Surface Modified Anticancer Nanoparticles;” 5,401,492 for “Water Insoluble Non-Magnetic Manganese Particles as Magnetic Resonance Enhancement Agents;” 5,429,824 for “Use of Tyloxapol as a Nanoparticulate Stabilizer;” 5,447,710 for “Method for Making Nanoparticulate X-Ray Blood Pool Contrast Agents Using High Molecular Weight Non-ionic Surfactants;” 5,451,393 for “X-Ray Contrast Compositions Useful in Medical Imaging;” 5,466,440 for “Formulations of Oral Gastrointestinal Diagnostic X-Ray Contrast Agents in Combination with Pharmaceutically Acceptable Clays;” 5,470,583 for “Method of Preparing Nanoparticle Compositions Containing Charged Phospholipids to Reduce Aggregation;” 5,472,683 for “Nanoparticulate Diagnostic Mixed Carbamic Anhydrides as X-Ray Contrast Agents for Blood Pool and Lymphatic System Imaging;” 5,500,204 for “Nanoparticulate Diagnostic Dimers as X-Ray Contrast Agents for Blood Pool and Lymphatic System Imaging;” 5,518,738 for “Nanoparticulate NSAID Formulations;” 5,521,218 for “Nanoparticulate Iododipamide Derivatives for Use as X-Ray Contrast Agents;” 5,525,328 for “Nanoparticulate Diagnostic Diatrizoxy Ester X-Ray Contrast Agents for Blood Pool and Lymphatic System Imaging;” 5,543,133 for “Process of Preparing X-Ray Contrast Compositions Containing Nanoparticles;” 5,552,160 for “Surface Modified NSAID Nanoparticles;” 5,560,931 for “Formulations of Compounds as Nanoparticulate Dispersions in Digestible Oils or Fatty Acids;” 5,565,188 for “Polyalkylene Block Copolymers as Surface Modifiers for Nanoparticles;” 5,569,448 for “Sulfated Non-ionic Block Copolymer Surfactant as Stabilizer Coatings for Nanoparticle Compositions;” 5,571,536 for “Formulations of Compounds as Nanoparticulate Dispersions in Digestible Oils or Fatty Acids;” 5,573,749 for “Nanoparticulate Diagnostic Mixed Carboxylic Anydrides as X-Ray Contrast Agents for Blood Pool and Lymphatic System Imaging;” 5,573,750 for “Diagnostic Imaging X-Ray Contrast Agents;” 5,573,783 for “Redispersible Nanoparticulate Film Matrices With Protective Overcoats;” 5,580,579 for “Site-specific Adhesion Within the GI Tract Using Nanoparticles Stabilized by High Molecular Weight, Linear Poly(ethylene Oxide) Polymers;” 5,585,108 for “Formulations of Oral Gastrointestinal Therapeutic Agents in Combination with Pharmaceutically Acceptable Clays;” 5,587,143 for “Butylene Oxide-Ethylene Oxide Block Copolymers Surfactants as Stabilizer Coatings for Nanoparticulate Compositions;” 5,591,456 for “Milled Naproxen with Hydroxypropyl Cellulose as Dispersion Stabilizer;” 5,593,657 for “Novel Barium Salt Formulations Stabilized by Non-ionic and Anionic Stabilizers;” 5,622,938 for “Sugar Based Surfactant for Nanocrystals;” 5,628,981 for “Improved Formulations of Oral Gastrointestinal Diagnostic X-Ray Contrast Agents and Oral Gastrointestinal Therapeutic Agents;” 5,643,552 for “Nanoparticulate Diagnostic Mixed Carbonic Anhydrides as X-Ray Contrast Agents for Blood Pool and Lymphatic System Imaging;” 5,718,388 for “Continuous Method of Grinding Pharmaceutical Substances;” 5,718,919 for “Nanoparticles Containing the R(−)Enantiomer of Ibuprofen;” 5,747,001 for “Aerosols Containing Beclomethasone Nanoparticle Dispersions;” 5,834,025 for “Reduction of Intravenously Administered Nanoparticulate Formulation Induced Adverse Physiological Reactions;” 6,045,829 “Nanocrystalline Formulations of Human Immunodeficiency Virus (HIV) Protease Inhibitors Using Cellulosic Surface Stabilizers;” 6,068,858 for “Methods of Making Nanocrystalline Formulations of Human Immunodeficiency Virus (HIV) Protease Inhibitors Using Cellulosic Surface Stabilizers;” 6,153,225 for “Injectable Formulations of Nanoparticulate Naproxen;” 6,165,506 for “New Solid Dose Form of Nanoparticulate Naproxen;” 6,221,400 for “Methods of Treating Mammals Using Nanocrystalline Formulations of Human Immunodeficiency Virus (HIV) Protease Inhibitors;” 6,264,922 for “Nebulized Aerosols Containing Nanoparticle Dispersions;” 6,267,989 for “Methods for Preventing Crystal Growth and Particle Aggregation in Nanoparticle Compositions;” 6,270,806 for “Use of PEG-Derivatized Lipids as Surface Stabilizers for Nanoparticulate Compositions;” 6,316,029 for “Rapidly Disintegrating Solid Oral Dosage Form,” 6,375,986 for “Solid Dose Nanoparticulate Compositions Comprising a Synergistic Combination of a Polymeric Surface Stabilizer and Dioctyl Sodium Sulfosuccinate;” 6,428,814 for “Bioadhesive Nanoparticulate Compositions Having Cationic Surface Stabilizers;” 6,431,478 for “Small Scale Mill;” and 6,432,381 for “Methods for Targeting Drug Delivery to the Upper and/or Lower Gastrointestinal Tract,” 6,592,903 for “Nanoparticulate Dispersions Comprising a Synergistic Combination of a Polymeric Surface Stabilizer and Dioctyl Sodium Sulfosuccinate,” 6,582,285 for “Apparatus for sanitary wet milling;” 6,656,504 for “Nanoparticulate Compositions Comprising Amorphous Cyclosporine;” 6,742,734 for “System and Method for Milling Materials;” 6,745,962 for “Small Scale Mill and Method Thereof;” 6,811,767 for “Liquid droplet aerosols of nanoparticulate drugs;” and 6,908,626 for “Compositions having a combination of immediate release and controlled release characteristics;” all of which are specifically incorporated by reference. In addition, U.S. Patent Application No. 20020012675 A1, published on Jan. 31, 2002, for “Controlled Release Nanoparticulate Compositions,” and WO 02/098565 for “System and Method for Milling Materials,” describe nanoparticulate compositions, and are specifically incorporated by reference.

Amorphous small particle compositions are described, for example, in U.S. Pat. Nos. 4,783,484 for “Particulate Composition and Use Thereof as Antimicrobial Agent;” 4,826,689 for “Method for Making Uniformly Sized Particles from Water-Insoluble Organic Compounds;” 4,997,454 for “Method for Making Uniformly-Sized Particles From Insoluble Compounds;” 5,741,522 for “Ultrasmall, Non-aggregated Porous Particles of Uniform Size for Entrapping Gas Bubbles Within and Methods;” and 5,776,496, for “Ultrasmall Porous Particles for Enhancing Ultrasound Back Scatter.”

### SUMMARY OF THE INVENTION

The present invention relates to nanoparticulate compositions comprising a benzothiophene, preferably raloxifene hydrochloride. The compositions comprise a benzothiophene, preferably raloxifene hydrochloride, and at least one surface stabilizer adsorbed on or associated with the surface of the benzothiophene particles. The nanoparticulate benzothiophene, preferably raloxifene hydrochloride, particles have an effective average particle size of less than about 2000 nm. A preferred dosage form of the invention is a solid dosage form, although any pharmaceutically acceptable dosage form can be utilized.

Another aspect of the invention is directed to pharmaceutical compositions comprising a nanoparticulate benzothiophene, preferably raloxifene hydrochloride, composition of the invention. The pharmaceutical compositions comprise a benzothiophene, preferably raloxifene hydrochloride, at least one surface stabilizer, and a pharmaceutically acceptable carrier, as well as any desired excipients.

Another aspect of the invention is directed to a nanoparticulate benzothiophene, preferably raloxifene hydrochloride, composition having improved pharmacokinetic profiles as compared to conventional microcrystalline or solubilized benzothiophene formulations.

In yet another embodiment, the invention encompasses a benzothiophene, preferably raloxifene hydrochloride, composition, wherein administration of the composition to a subject in a fasted state is bioequivalent to administration of the composition to a subject in a fed state.

Another embodiment of the invention is directed to nanoparticulate benzothiophene, preferably raloxifene hydrochloride, compositions additionally comprising one or more compounds useful in treating osteoporosis, breast cancer, or related conditions.

This invention further discloses a method of making a nanoparticulate benzothiophene, preferably raloxifene hydrochloride, composition according to the invention. Such a method comprises contacting a benzothiophene, preferably raloxifene hydrochloride, and at least one surface stabilizer for a time and under conditions sufficient to provide a nanoparticulate benzothiophene composition, and preferably a raloxifene hydrochloride composition. The one or more surface stabilizers can be contacted with a benzothiophene, preferably raloxifene hydrochloride, either before, during, or after size reduction of the benzothiophene.

The present invention is also directed to methods of treatment using the nanoparticulate benzothiophene, preferably raloxifene hydrochloride, compositions of the invention for conditions such as osteoporosis, carcinomas of the breast and lymph glands, and the like.

Both the foregoing general description and the following detailed description are exemplary and explanatory and are intended to provide further explanation of the invention as claimed. Other objects, advantages, and novel features will be readily apparent to those skilled in the art from the following detailed description of the invention.

### DETAILED DESCRIPTION OF THE INVENTION

### A. Introduction

The present invention is directed to nanoparticulate compositions comprising a benzothiophene, preferably raloxifene hydrochloride. The compositions comprise a benzothiophene, preferably raloxifene hydrochloride, and preferably at least one surface stabilizer adsorbed on or associated with the surface of the drug. The nanoparticulate benzothiophene, preferably raloxifene hydrochloride, particles have an effective average particle size of less than about 2000 nm.

Advantages of a nanoparticulate benzothiophene, preferably a nanoparticulate raloxifene hydrochloride, formulation of the invention include, but are not limited to: (1) smaller tablet or other solid dosage form size, or less frequent administration of the formulation; (2) smaller doses of drug required to obtain the same pharmacological effect as compared to conventional microcrystalline or solubilized forms of a benzothiophene; (3) increased bioavailability as compared to conventional microcrystalline or solubilized forms of a benzothiophene; (4) improved pharmacokinetic profiles, such as Tmax, Cmax, and AUC profiles as compared to conventional microcrystalline or solubilized forms of a benzothiophene; (5) substantially similar pharmacokinetic profiles of the nanoparticulate benzothiophene compositions when administered in the fed versus the fasted state; (6) bioequivalent pharmacokinetic profiles of the nanoparticulate benzothiophene compositions when administered in the fed versus the fasted state; (7) an increased rate of dissolution for the nanoparticulate benzothiophene compositions as compared to conventional microcrystalline or solubilized forms of the same benzothiophene; (8) bioadhesive benzothiophene compositions; and (9) use of the nanoparticulate benzothiophene compositions in conjunction with other active agents useful in treating osteoporosis, carcinomas of the breast and lymph glands and, related conditions.

The present invention also includes nanoparticulate benzothiophene, preferably nanoparticulate raloxifene hydrochloride compositions, together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like.

A preferred dosage form of the invention is a solid dosage form, although any pharmaceutically acceptable dosage form can be utilized. Exemplary solid dosage forms include, but are not limited to, tablets, capsules, sachets, lozenges, powders, pills, or granules, and the solid dosage form can be, for example, a fast melt dosage form, controlled release dosage form, lyophilized dosage form, delayed release dosage form, extended release dosage form, pulsatile release dosage form, mixed immediate release and controlled release dosage form, or a combination thereof. A solid dose tablet formulation is preferred.

### B. Definitions

The present invention is described herein using several definitions, as set forth below and throughout the application.

The term “effective average particle size”, as used herein means that at least 50% of the nanoparticulate benzothiophene, or preferably raloxifene hydrochloride particles, have a weight average size of less than about 2000 nm, when measured by, for example, sedimentation field flow fractionation, photon correlation spectroscopy, light scattering, disk centrifugation, and other techniques known to those of skill in the art.

As used herein, “about” will be understood by persons of ordinary skill in the art and will vary to some extent on the context in which it is used. If there are uses of the term which are not clear to persons of ordinary skill in the art given the context in which it is used, “about” will mean up to plus or minus 10% of the particular term.

As used herein with reference to a stable benzothiophene or a stable raloxifene hydrochloride particle connotes, but is not limited to one or more of the following parameters: (1), benzothiophene or raloxifene hydrochloride particles do not appreciably flocculate or agglomerate due to interparticle attractive forces or otherwise significantly increase in particle size over time; (2) that the physical structure of the benzothiophene or raloxifene hydrochloride particles is not altered over time, such as by conversion from an amorphous phase to a crystalline phase; (3) that the benzothiophene or raloxifene hydrochloride particles are chemically stable; and/or (4) where the benzothiophene or raloxifene hydrochloride has not been subject to a heating step at or above the melting point of the benzothiophene or raloxifene hydrochloride in the preparation of the nanoparticles of the present invention.

The term “conventional” or “non-nanoparticulate active agent” shall mean an active agent which is solubilized or which has an effective average particle size of greater than about 2000 nm. Nanoparticulate active agents as defined herein have an effective average particle size of less than about 2000 nm.

The phrase “poorly water soluble drugs” as used herein refers to those drugs that have a solubility in water of less than about 30 mg/ml, preferably less than about 20 mg/ml, preferably less than about 10 mg/ml, or preferably less than about 1 mg/ml.

As used herein, the phrase “therapeutically effective amount” shall mean that drug dosage that provides the specific pharmacological response for which the drug is administered in a significant number of subjects in need of such treatment. It is emphasized that a therapeutically effective amount of a drug that is administered to a particular subject in a particular instance will not always be effective in treating the conditions/diseases described herein, even though such dosage is deemed to be a therapeutically effective amount by those of skill in the art.

### C. The Nanoparticulate Composition

There are a number of enhanced pharmacological characteristics of nanoparticulate benzothiophene compositions of the present invention.

1. Increased Bioavailability

The benzothiophene formulations of the present invention, preferably raloxifene hydrochloride formulations of the invention, exhibit increased bioavailability at the same dose of the same benzothiophene, and require smaller doses as compared to prior conventional benzothiophene formulations, including conventional raloxifene hydrochloride formulations. Thus, a nanoparticulate raloxifene hydrochloride tablet, if administered to a patient in a fasted state is not bioequivalent to administration of a conventional microcrystalline raloxifene hydrochloride tablet in a fasted state.

The non-bioequivalence is significant because it means that the nanoparticulate raloxifene hydrochloride dosage form exhibits significantly greater drug absorption. And for the nanoparticulate raloxifene hydrochloride dosage form to be bioequivalent to the conventional microcrystalline raloxifene hydrochloride dosage form, the nanoparticulate raloxifene hydrochloride dosage form would have to contain significantly less drug. Thus, the nanoparticulate raloxifene hydrochloride dosage form significantly increases the bioavailability of the drug.

Moreover, a nanoparticulate raloxifene hydrochloride dosage form requires less drug to obtain the same pharmacological effect observed with a conventional microcrystalline raloxifene hydrochloride dosage form (e.g., EVISTA®). Therefore, the nanoparticulate raloxifene hydrochloride dosage form has an increased bioavailability as compared to the conventional microcrystalline raloxifene hydrochloride dosage form.

2. The Pharmacokinetic Profiles of the Benzothiophene Compositions of the Invention are not Affected by the Fed or Fasted State of the Subject Ingesting the Compositions

The compositions of the present invention encompass a benzothiophene, preferably raloxifene hydrochloride, wherein the pharmacokinetic profile of the benzothiophene is not substantially affected by the fed or fasted state of a subject ingesting the composition. This means that there is little or no appreciable difference in the quantity of drug absorbed or the rate of drug absorption when the nanoparticulate benzothiophene, preferably raloxifene hydrochloride, compositions are administered in the fed versus the fasted state.

Benefits of a dosage form which substantially eliminates the effect of food include an increase in subject convenience, thereby increasing subject compliance, as the subject does not need to ensure that they are taking a dose either with or without food. This is significant, as with poor subject compliance an increase in the medical condition for which the drug is being prescribed may be observed, i.e., osteoporosis or cardiovascular problems for poor subject compliance with a benzothiophene such as raloxifene hydrochloride.

The invention also preferably provides benzothiophene compositions, such as raloxifene hydrochloride compositions, having a desirable pharmacokinetic profile when administered to mammalian subjects. The desirable pharmacokinetic profile of the benzothiophene compositions preferably includes, but is not limited to: (1) a Cₘₐₓ for benzothiophene, when assayed in the plasma of a mammalian subject following administration, that is preferably greater than the Cₘₐₓ for a non-nanoparticulate benzothiophene formulation (e.g., EVISTA®), administered at the same dosage; and/or (2) an AUC for benzothiophene, when assayed in the plasma of a mammalian subject following administration, that is preferably greater than the AUC for a non-nanoparticulate benzothiophene formulation (e.g., EVISTA®), administered at the same dosage; and/or (3) a Tmax for benzothiophene, when assayed in the plasma of a mammalian subject following administration, that is preferably less than the Tmax for a non-nanoparticulate benzothiophene formulation (e.g., EVISTA®), administered at the same dosage. The desirable pharmacokinetic profile, as used herein, is the pharmacokinetic profile measured after the initial dose of benzothiophene.

In one embodiment, a preferred benzothiophene composition of the invention is a nanoparticulate raloxifene hydrochloride composition that exhibits in comparative pharmacokinetic testing with a non-nanoparticulate benzothiophene formulation (e.g., EVISTA®), administered at the same dosage, a Tₘₐₓ not greater than about 90%, not greater than about 80%, not greater than about 70%, not greater than about 60%, not greater than about 50%, not greater than about 30%, not greater than about 25%, not greater than about 20%, not greater than about 15%, not greater than about 10%, or not greater than about 5% of the Tₘₐₓ exhibited by the non-nanoparticulate benzothiophene formulation.

In another embodiment, the benzothiophene composition of the invention is a nanoparticulate raloxifene hydrochloride composition that exhibits in comparative pharmacokinetic testing with a non-nanoparticulate benzothiophene formulation of (e.g., EVISTA®), administered at the same dosage, a Cₘₐₓ which is at least about 50%, at least about 100%, at least about 200%, at least about 300%, at least about 400%, at least about 500%, at least about 600%, at least about 700%, at least about 800%, at least about 900%, at least about 1000%, at least about 1100%, at least about 1200%, at least about 1300%, at least about 1400%, at least about 1500%, at least about 1600%, at least about 1700%, at least about 1800%, or at least about 1900% greater than the Cₘₐₓ exhibited by the non-nanoparticulate benzothiophene formulation.

In yet another embodiment, the benzothiophene composition of the invention is a raloxifene hydrochloride nanoparticulate composition exhibits in comparative pharmacokinetic testing with a non-nanoparticulate benzothiophene formulation (e.g., EVISTA®), administered at the same dosage, an AUC which is at least about 25%, at least about 50%, at least about 75%, at least about 100%, at least about 125%, at least about 150%, at least about 175%, at least about 200%, at least about 225%, at least about 250%, at least about 275%, at least about 300%, at least about 350%, at least about 400%, at least about 450%, at least about 500%, at least about 550%, at least about 600%, at least about 750%, at least about 700%, at least about 750%, at least about 800%, at least about 850%, at least about 900%, at least about 950%, at least about 1000%, at least about 1050%, at least about 1100%, at least about 1150%, or at least about 1200% greater than the AUC exhibited by the non-nanoparticulate benzothiophene formulation (e.g., EVISTA®).

3. Bioequivalency of the Benzothiophene Compositions of the Invention when Administered in the Fed Versus the Fasted State

The invention also encompasses a composition comprising a nanoparticulate benzothiophene, preferably a nanoparticulate raloxifene hydrochloride, in which administration of the composition to a subject in a fasted state is bioequivalent to administration of the composition to a subject in a fed state.

The difference in absorption of the compositions comprising the nanoparticulate benzothiophene or preferably, the nanoparticulate raloxifene hydrochloride when administered in the fed versus the fasted state, is preferably less than about 35%, less than about 30%, less than about 25%, less than about 20%, less than about 15%, less than about 10%, less than about 5%, or less than about 3%.

In one embodiment of the invention, the invention encompasses nanoparticulate benzothiophene or preferably, the nanoparticulate raloxifene hydrochloride, wherein administration of the composition to a subject in a fasted state is bioequivalent to administration of the composition to a subject in a fed state, in particular as defined by Cₘₐₓ and AUC guidelines given by the U.S. Food and Drug Administration and the corresponding European regulatory agency (EMEA). Under U.S. FDA guidelines, two products or methods are bioequivalent if the 90% Confidence Intervals (CI) for AUC and Cₘₐₓ are between 0.80 to 1.25 (Tₘₐₓ measurements are not relevant to bioequivalence for regulatory purposes). To show bioequivalency between two compounds or administration conditions pursuant to Europe's EMEA guidelines, the 90% CI for AUC must be between 0.80 to 1.25 and the 90% CI for Cₘₐₓ must between 0.70 to 1.43.

4. Dissolution Profiles of the Benzothiophene Compositions of the Invention

The benzothiophene compositions of the present invention have unexpectedly dramatic dissolution profiles. Rapid dissolution of an administered active agent is preferable, as faster dissolution generally leads to faster onset of action and greater bioavailability. To improve the dissolution profile and bioavailability of benzothiophenes, and raloxifene hydrochloride in particular, it is useful to increase the drug's dissolution so that it could attain a level close to 100%.

The benzothiophene compositions of the present invention, including raloxifene hydrochloride compositions, preferably have a dissolution profile in which within about 5 minutes at least about 20% of the composition is dissolved. In other embodiments of the invention, at least about 30% or about 40% of the benzothiophene or raloxifene hydrochloride composition is dissolved within about 5 minutes. In yet other embodiments of the invention, preferably at least about 40%, about 50%, about 60%, about 70%, or about 80% of the benzothiophene composition, or preferably the raloxifene hydrochloride composition is dissolved within about 10 minutes. Finally, in another embodiment of the invention, preferably at least about 70%, about 80%, about 90%, or about 100% of the benzothiophene composition, or preferably, the raloxifene hydrochloride composition is dissolved within about 20 minutes.

Dissolution is preferably measured in a medium which is discriminating. Such a dissolution medium will produce two very different dissolution curves for two products having very different dissolution profiles in gastric juices, i.e., the dissolution medium is predictive of in vivo dissolution of a composition. An exemplary dissolution medium is an aqueous medium containing the surfactant sodium lauryl sulfate at 0.025 M. Determination of the amount dissolved can be carried out by spectrophotometry. The rotating blade method (European Pharmacopoeia) can be used to measure dissolution.

5. Redispersibility Profiles of the Benzothiophene Compositions of the Invention

An additional feature of the benzothiophene compositions of the present invention is that the compositions redisperse such that the effective average particle size of the redispersed benzothiophene particles is less than about 2 microns. This is significant, as if upon administration the nanoparticulate benzothiophene compositions of the invention did not redisperse to a nanoparticulate particle size, then the dosage form may lose the benefits afforded by formulating the benzothiophene into a nanoparticulate particle size. A nanoparticulate size suitable for the present invention is an effective average particle size of less than about 2000 nm.

Indeed, the nanoparticulate active agent compositions of the present invention benefit from the small particle size of the active agent; if the active agent does not redisperse into a small particle size upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form may fall well below that observed with the liquid dispersion form of the nanoparticulate active agent.

In other embodiments of the invention, the redispersed benzothiophene, preferably raloxifene hydrochloride, particles of the invention have an effective average particle size of less than about less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. Such methods suitable for measuring effective average particle size are known to a person of ordinary skill in the art.

6. Other Pharmaceutical Excipients

Pharmaceutical compositions according to the invention may also comprise one or more binding agents, filling agents, lubricating agents, suspending agents, sweeteners, flavoring agents, preservatives, buffers, wetting agents, disintegrants, effervescent agents, and other excipients. Such excipients are known in the art.

Examples of filling agents are lactose monohydrate, lactose anhydrous, and various starches; examples of binding agents are various celluloses and cross-linked polyvinylpyrrolidone, microcrystalline cellulose, such as Avicel® PH101 and Avicel® PH102, microcrystalline cellulose, and silicified microcrystalline cellulose (ProSolv SMCC™).

Suitable lubricants, including agents that act on the flowability of the powder to be compressed, are colloidal silicon dioxide, such as Aerosil® 200, talc, stearic acid, magnesium stearate, calcium stearate, and silica gel.

Examples of sweeteners are any natural or artificial sweetener, such as sucrose, xylitol, sodium saccharin, cyclamate, aspartame, and acsulfame. Examples of flavoring agents are Magnasweet® (trademark of MAFCO), bubble gum flavor, and fruit flavors, and the like.

Examples of preservatives are potassium sorbate, methylparaben, propylparaben, benzoic acid and its salts, other esters of parahydroxybenzoic acid such as butylparaben, alcohols such as ethyl or benzyl alcohol, phenolic compounds such as phenol, or quarternary compounds such as benzalkonium chloride.

Suitable diluents include pharmaceutically acceptable inert fillers, such as microcrystalline cellulose, lactose, dibasic calcium phosphate, saccharides, and/or mixtures of any of the foregoing. Examples of diluents include microcrystalline cellulose, such as Avicel® PH101 and Avicel® PH102; lactose such as lactose monohydrate, lactose anhydrous, and Pharmatose® DCL21; dibasic calcium phosphate such as Emcompress®; mannitol; starch; sorbitol; sucrose; and glucose.

Suitable disintegrants include lightly crosslinked polyvinyl pyrrolidone, corn starch, potato starch, maize starch, and modified starches, croscarmellose sodium, cross-povidone, sodium starch glycolate, and mixtures thereof.

Examples of effervescent agents are effervescent couples such as an organic acid and a carbonate or bicarbonate. Suitable organic acids include, for example, citric, tartaric, malic, fumaric, adipic, succinic, and alginic acids and anhydrides and acid salts. Suitable carbonates and bicarbonates include, for example, sodium carbonate, sodium bicarbonate, potassium carbonate, potassium bicarbonate, magnesium carbonate, sodium glycine carbonate, L-lysine carbonate, and arginine carbonate. Alternatively, only the sodium bicarbonate component of the effervescent couple may be present.

7. Combination Pharmacokinetic Profile Compositions

In yet another embodiment of the invention, a first nanoparticulate benzothiophene composition, preferably a raloxifene hydrochloride composition, providing a desired pharmacokinetic profile is co-administered, sequentially administered, or combined with at least one other benzothiophene composition, preferably a raloxifene hydrochloride composition, that generates a desired different pharmacokinetic profile. More than two benzothiophene compositions, preferably raloxifene hydrochloride compositions, can be co-administered, sequentially administered, or combined. While the first benzothiophene composition, preferably raloxifene hydrochloride composition, has a nanoparticulate particle size, the additional one or more benzothiophene compositions can be nanoparticulate, solubilized, or have a microparticulate particle size.

The second, third, fourth, etc., benzothiophene compositions can differ from the first, and from each other, for example: (1) in the effective average particle sizes of benzothiophene; or (2) in the dosage of benzothiophene. Such a combination composition can reduce the dose frequency required.

If the second benzothiophene composition has a nanoparticulate particle size, then preferably the benzothiophene particles of the second composition have at least one surface stabilizer associated with the surface of the drug particles. The one or more surface stabilizers can be the same as or different from the surface stabilizer(s) present in the first benzothiophene composition.

Preferably where co-administration of a “fast-acting” formulation and a “longer-lasting” formulation is desired, the two formulations are combined within a single composition, for example a dual-release composition.

8. Benzothiophene Compositions Used in Conjunction with Other Active Agents

The benzothiophene, preferably a raloxifene hydrochloride, compositions of the invention can additionally comprise one or more compounds useful in treating osteoporosis, breast cancer, or related conditions. The compositions of the invention can be co-formulated with such other active agents, or the compositions of the invention can be co-administered or sequentially administered in conjunction with such active agents.

Examples of active agents useful in treating osteoporosis or related conditions, such as Paget's disease, include, but are not limited to, calcium supplements, vitamin D, bisphosphonates, bone formation agents, estrogens, parathyroid hormones and selective receptor modulators. Specific examples of drugs include, but are not limited to, risedronate sodium (Actonel®), ibandronate sodium (Boniva®), etidronate Disodium (Didronel®), parathyroid hormone and derivatives thereof, such as teriparatide (Forteo®), alendronate (Fosamax®), and calcitonin (Miacalcin®).

Breast cancer drugs include, but are not limited to, chemotherapy regimens, paclitaxel (Abraxane® or Taxol®), doxorubicin (Adriamycin®), pamidronate disodium (Aredia®), anastrozole (Arimidex®), exemestane (Aromasin®), cyclophosphamide (Cytoxan®), epirubicin (Ellence®), toremifene (Fareston®), letrozole (Femara®), trastuzumab (Herceptin®), megestrol (Megace®), Nolvadex (Tamoxifen®), docetaxel (Taxotere®), capecitabine (Xeloda®), goserelin acetate (Zoladex®), and zoledronic acid (Zometa®). Examples of chemotherapy combinations used to treat breast cancer include: (1) cyclophosphamide (Cytoxan®), methotrexate (Amethopterin®, Mexate®, Folex®), and fluorouracil (Fluorouracil®, 5-Fu®, Adrucil®) (this therapy is called CMF); (2) cyclophosphamide, doxorubicin (Adriamycin®), and fluorouracil (this therapy is called CAF); (3) doxorubicin (Adriamycin®) and cyclophosphamide (this therapy is called AC); (4) doxorubicin (Adriamycin®) and cyclophosphamide with paclitaxel (Taxol®); (4) doxorubicin (Adriamycin®), followed by CMF; and (5) cyclophosphamide, epirubicin (Ellence®), and fluorouracil.

### D. Compositions

The invention provides compositions comprising nanoparticulate benzothiophene, preferably a raloxifene hydrochloride, particles and at least one surface stabilizer. The surface stabilizers are preferably adsorbed to or associated with the surface of the benzothiophene particles. Surface stabilizers useful herein do not chemically react with the benzothiophene particles or itself. Preferably, individual molecules of the surface stabilizer are essentially free of intermolecular cross-linkages. The compositions can comprise two or more surface stabilizers.

The present invention also includes nanoparticulate benzothiophene compositions together with one or more non-toxic physiologically acceptable carriers, adjuvants, or vehicles, collectively referred to as carriers. The compositions can be formulated for parenteral injection (e.g., intravenous, intramuscular, or subcutaneous), oral administration in solid, liquid, or aerosol form, vaginal, nasal, rectal, ocular, local (powders, ointments or drops), buccal, intracisternal, intraperitoneal, or topical administration, and the like.

1. Benzothiophene

Benzothiophene or a salt thereof, preferably raloxifene hydrochloride, can be in a crystalline phase, an amorphous phase, a semi-crystalline phase, a semi-amorphous phase, or a mixtures thereof.

The benzothiophene or a salt thereof, preferably raloxifene hydrochloride, of the invention is poorly soluble and dispersible in at least one liquid media. A preferred dispersion media is water. The dispersion media can be, for example, water, safflower oil, ethanol, t-butanol, glycerin, polyethylene glycol (PEG), hexane, or glycol.

The benzothiophene or a salt thereof, preferably raloxifene hydrochloride active compounds, useful in the current invention can also be made according to established procedures, such as those detailed in U.S. Pat. Nos. 4,133,814 to Jones et al; 4,418,068 and 4,380,635 to Peters; and European Patent Application 95306050.6, Publication No. 0699672, Kjell, et al., filed Aug. 30, 1995, published Mar. 6, 1996, all of which are incorporated by reference herein. In general, the process starts with a benzo[b]thiophene having a 6-hydroxyl group and a 2-(4-hydroxyphenyl) group. The starting compound is protected, acylated, and deprotected to form the formula I compounds. Examples of the preparation of such compounds are provided in the U.S. patents discussed above.

2. Surface Stabilizers

Preferably, the nanoparticulate raloxifene hydrochloride compositions of the present invention comprise the active raloxifene hydrochloride nanoparticles that is combined with a surface stabilizer, and combinations of more than one surface stabilizer can be used in the present invention.

Useful surface stabilizers which can be employed in the invention include, but are not limited to, known organic and inorganic pharmaceutical excipients. Such excipients include various polymers, low molecular weight oligomers, natural products, and surfactants. Surface stabilizers include nonionic, anionic, cationic, ionic, and zwitterionic surfactants.

Representative examples of surface stabilizers include hydroxypropyl methylcellulose (now known as hypromellose), hydroxypropylcellulose, polyvinylpyrrolidone, sodium lauryl sulfate, dioctylsulfosuccinate, gelatin, casein, lecithin (phosphatides), dextran, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers (e.g., macrogol ethers such as cetomacrogol 1000), polyoxyethylene castor oil derivatives, polyoxyethylene sorbitan fatty acid esters (e.g., the commercially available Tweens® such as e.g., Tween 20® and Tween 80® (ICI Speciality Chemicals)); polyethylene glycols (e.g., Carbowaxs 3550® and 934® (Union Carbide)), polyoxyethylene stearates, colloidal silicon dioxide, phosphates, carboxymethylcellulose calcium, carboxymethylcellulose sodium, methylcellulose, hydroxyethylcellulose, hypromellose phthalate, noncrystalline cellulose, magnesium aluminium silicate, triethanolamine, polyvinyl alcohol (PVA), 4-(1,1,3,3-tetramethylbutyl)-phenol polymer with ethylene oxide and formaldehyde (also known as tyloxapol, superione, and triton), poloxamers (e.g., Pluronics F68′ and F108®, which are block copolymers of ethylene oxide and propylene oxide); poloxamines (e.g., Tetronic 908®, also known as Poloxamine 908®, which is a tetrafunctional block copolymer derived from sequential addition of propylene oxide and ethylene oxide to ethylenediamine (BASF Wyandotte Corporation, Parsippany, N.J.)); Tetronic 1508® (T-1508) (BASF Wyandotte Corporation), Tritons X-200®, which is an alkyl aryl polyether sulfonate (Rohm and Haas); Crodestas F-110®, which is a mixture of sucrose stearate and sucrose distearate (Croda Inc.); p-isononylphenoxypoly-(glycidol), also known as Olin-IOG® or Surfactant 110-G® (Olin Chemicals, Stamford, Conn.); Crodestas SL-40® (Croda, Inc.); and SA9OHCO, which is C₁₈H₃₇CH₂(CON(CH₃)—CH₂(CHOH)₄(CH₂0H)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; PEG-phospholipid, PEG-cholesterol, PEG-cholesterol derivative, PEG-vitamin A, PEG-vitamin E, lysozyme, random copolymers of vinyl pyrrolidone and vinyl acetate, such as Plasdone® S630, and the like.

Examples of useful cationic surface stabilizers include, but are not limited to, polymers, biopolymers, polysaccharides, cellulosics, alginates, phospholipids, and nonpolymeric compounds, such as zwitterionic stabilizers, poly-n-methylpyridinium, anthryul pyridinium chloride, cationic phospholipids, chitosan, polylysine, polyvinylimidazole, polybrene, polymethylmethacrylate trimethylammoniumbromide bromide (PMMTMABr), hexyldesyltrimethylammonium bromide (HDMAB), and polyvinylpyrrolidone-2-dimethylaminoethyl methacrylate dimethyl sulfate.

Other useful cationic stabilizers include, but are not limited to, cationic lipids, sulfonium, phosphonium, and quarternary ammonium compounds, such as stearyltrimethylammonium chloride, benzyl-di(2-chloroethyl)ethylammonium bromide, coconut trimethyl ammonium chloride or bromide, coconut methyl dihydroxyethyl ammonium chloride or bromide, decyl triethyl ammonium chloride, decyl dimethyl hydroxyethyl ammonium chloride or bromide, C₁₂₋₁₅-dimethyl hydroxyethyl ammonium chloride or bromide, coconut dimethyl hydroxyethyl ammonium chloride or bromide, myristyl trimethyl ammonium methyl sulphate, lauryl dimethyl benzyl ammonium chloride or bromide, lauryl dimethyl (ethenoxy)₄ ammonium chloride or bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts and dialkyl-dimethylammonium salts, lauryl trimethyl ammonium chloride, ethoxylated alkyamidoalkyldialkylammonium salt and/or an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride and dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂, C₁₅, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride (ALIQUAT 336™), POLYQUAT 10™, tetrabutylammonium bromide, benzyl trimethylammonium bromide, choline esters (such as choline esters of fatty acids), benzalkonium chloride, stearalkonium chloride compounds (such as stearyltrimonium chloride and Di-stearyldimonium chloride), cetyl pyridinium bromide or chloride, halide salts of quaternized polyoxyethylalkylamines, MIRAPOL™ and ALKAQUAT™ (Alkaril Chemical Company), alkyl pyridinium salts; amines, such as alkylamines, dialkylamines, alkanolamines, polyethylenepolyamines, N,N-dialkylaminoalkyl acrylates, and vinyl pyridine, amine salts, such as lauryl amine acetate, stearyl amine acetate, alkylpyridinium salt, and alkylimidazolium salt, and amine oxides; imide azolinium salts; protonated quaternary acrylamides; methylated quaternary polymers, such as poly[diallyl dimethylammonium chloride] and poly-[N-methyl vinyl pyridinium chloride]; and cationic guar.

Such exemplary cationic surface stabilizers and other useful cationic surface stabilizers are described in J. Cross and E. Singer, Cationic Surfactants: Analytical and Biological Evaluation (Marcel Dekker, 1994); P. and D. Rubingh (Editor), Cationic Surfactants: Physical Chemistry (Marcel Dekker, 1991); and J. Richmond, Cationic Surfactants: Organic Chemistry, (Marcel Dekker, 1990).

Nonpolymeric surface stabilizers are any nonpolymeric compound, such benzalkonium chloride, a carbonium compound, a phosphonium compound, an oxonium compound, a halonium compound, a cationic organometallic compound, a quarternary phosphorous compound, a pyridinium compound, an anilinium compound, an ammonium compound, a hydroxylammonium compound, a primary ammonium compound, a secondary ammonium compound, a tertiary ammonium compound, and quarternary ammonium compounds of the formula NR₁R₂R₃R₄⁽⁺⁾. For compounds of the formula NR₁R₂R₃R₄⁽⁺⁾:
(i) none of R₁-R₄ are CH₃;
(ii) one of R₁-R₄ is CH₃;
(iii) three of R₁-R₄ are CH₃;
(iv) all of R₁-R₄ are CH₃;
(v) two of R₁-R₄ are CH₃, one of R₁-R₄ is C₆H₅CH₂, and one of R₁-R₄ is an alkyl chain of seven carbon atoms or less;
(vi) two of R₁-R₄ are CH₃, one of R₁-R₄ is C₆H₅CH₂, and one of R₁-R₄ is an alkyl chain of nineteen carbon atoms or more;
(vii) two of R₁-R₄ are CH₃ and one of R₁-R₄ is the group C₆H₅(CH₂)ₙ, where n>1;
(viii) two of R₁-R₄ are CH₃, one of R₁-R₄ is C₆H₅CH₂, and one of R₁-R₄ comprises at least one heteroatom;
(ix) two of R₁-R₄ are CH₃, one of R₁-R₄ is C₆H₅CH₂, and one of R₁-R₄ comprises at least one halogen;
(x) two of R₁-R₄ are CH₃, one of R₁-R₄ is C₆H₅CH₂, and one of R₁-R₄ comprises at least one cyclic fragment;
(xi) two of R₁-R₄ are CH₃ and one of R₁-R₄ is a phenyl ring; or
(xii) two of R₁-R₄ are CH₃ and two of R₁-R₄ are purely aliphatic fragments.

Such compounds include, but are not limited to, behenalkonium chloride, benzethonium chloride, cetylpyridinium chloride, behentrimonium chloride, lauralkonium chloride, cetalkonium chloride, cetrimonium bromide, cetrimonium chloride, cethylamine hydrofluoride, chlorallylmethenamine chloride (Quaternium-15), distearyldimonium chloride (Quaternium-5), dodecyl dimethyl ethylbenzyl ammonium chloride (Quaternium-14), Quaternium-22, Quaternium-26, Quaternium-18 hectorite, dimethylaminoethylchloride hydrochloride, cysteine hydrochloride, diethanolammonium POE (10) oletyl ether phosphate, diethanolammonium POE (3)oleyl ether phosphate, tallow alkonium chloride, dimethyl dioctadecylammoniumbentonite, stearalkonium chloride, domiphen bromide, denatonium benzoate, myristalkonium chloride, laurtrimonium chloride, ethylenediamine dihydrochloride, guanidine hydrochloride, pyridoxine HCl, iofetamine hydrochloride, meglumine hydrochloride, methylbenzethonium chloride, myrtrimonium bromide, oleyltrimonium chloride, polyquaternium-1, procainehydrochloride, cocobetaine, stearalkonium bentonite, stearalkoniumhectonite, stearyl trihydroxyethyl propylenediamine dihydrofluoride, tallowtrimonium chloride, and hexadecyltrimethyl ammonium bromide.

The surface stabilizers are commercially available and/or can be prepared by techniques known in the art. Most of these surface stabilizers are known pharmaceutical excipients and are described in detail in the Handbook of Pharmaceutical Excipients, published jointly by the American Pharmaceutical Association and The Pharmaceutical Society of Great Britain (The Pharmaceutical Press, 2000), and is specifically incorporated herein by reference.

3. Nanoparticulate Benzothiophene Particle Size

The compositions of the present invention contain nanoparticulate benzothiophene particles, preferably nanoparticulate raloxifene hydrochloride particles, which have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods.

By “an effective average particle size of less than about 2000 nm” it is meant that at least 50% of the benzothiophene, preferably raloxifene hydrochloride, particles have a particle size of less than the effective average, by weight, i.e., less than about 2000 nm, 1900 nm, 1800 nm, etc. (as listed above), when measured by the above-noted techniques. Preferably, at least about 70%, at least about 90%, at least about 95%, or at least about 99% of the benzothiophene particles, preferably raloxifene hydrochloride particles, by weight, have a particle size of less than the effective average, i.e., less than about 2000 nm, 1900 nm, 1800 nm, 1700 nm, etc.

In the present invention, the value for D50 of a nanoparticulate benzothiophene composition, preferably a nanoparticulate raloxifene hydrochloride composition is the particle size below which 50% of the benzothiophene particles fall, by weight. Similarly, D90 is the particle size below which 90% of the benzothiophene particles fall, by weight, and D99 is the particle size below which 99% of the raloxifene hydrochloride particles fall, by weight.

4. Concentration of the Benzothiophene and Surface Stabilizers

The relative amounts of a benzothiophene, preferably raloxifene hydrochloride, and one or more surface stabilizers can vary widely. The optimal amount of the individual components can depend, for example, upon the particular benzothiophene selected, the hydrophilic lipophilic balance (HLB), melting point, and the surface tension of water solutions of the stabilizer, etc.

In one embodiment, the concentration of the benzothiophene, preferably raloxifene hydrochloride, can vary from about 99.5% to about 0.001%, from about 95% to about 0.1%, or from about 90% to about 0.5%, by weight, based on the total combined weight of the benzothiophene and at least one surface stabilizer, not including other excipients.

In another embodiment, the concentration of the at least one surface stabilizer can vary from about 0.5% to about 99.999%, from about 5.0% to about 99.9%, or from about 10% to about 99.5%, by weight, based on the total combined dry weight of the benzothiophene and at least one surface stabilizer, not including other excipients.

### E. Methods of Making Benzothiophene Formulations

In another aspect of the invention there is provided a method of preparing the nanoparticulate benzothiophene, preferably nanoparticulate raloxifene hydrochloride, formulations of the invention. The method comprises of one of the following methods: attrition, precipitation, evaporation, or combinations of these. Exemplary methods of making nanoparticulate compositions are described in U.S. Pat. No. 5,145,684. Methods of making nanoparticulate compositions are also described in U.S. Pat. No. 5,518,187 for “Method of Grinding Pharmaceutical Substances;” U.S. Pat. No. 5,718,388 for “Continuous Method of Grinding Pharmaceutical Substances;” U.S. Pat. No. 5,862,999 for “Method of Grinding Pharmaceutical Substances;” U.S. Pat. No. 5,665,331 for “Co-Microprecipitation of Nanoparticulate Pharmaceutical Agents with Crystal Growth Modifiers;” U.S. Pat. No. 5,662,883 for “Co-Microprecipitation of Nanoparticulate Pharmaceutical Agents with Crystal Growth Modifiers;” U.S. Pat. No. 5,560,932 for “Microprecipitation of Nanoparticulate Pharmaceutical Agents;” U.S. Pat. No. 5,543,133 for “Process of Preparing X-Ray Contrast Compositions Containing Nanoparticles;” U.S. Pat. No. 5,534,270 for “Method of Preparing Stable Drug Nanoparticles;” U.S. Pat. No. 5,510,118 for “Process of Preparing Therapeutic Compositions Containing Nanoparticles;” and U.S. Pat. No. 5,470,583 for “Method of Preparing Nanoparticle Compositions Containing Charged Phospholipids to Reduce Aggregation,” all of which are specifically incorporated by reference.

Following milling, homogenization, precipitation, etc., the resultant nanoparticulate benzothiophene, preferably nanoparticulate raloxifene hydrochloride, composition can be utilized a suitable dosage form for administration.

Preferably, the dispersion media used for the size reduction process is aqueous. However, any media in which benzothiophene, preferably raloxifene hydrochloride, is poorly soluble and dispersible can be used as a dispersion media. Non-aqueous examples of dispersion media include, but are not limited to, aqueous salt solutions, safflower oil and solvents such as ethanol, t-butanol, hexane, and glycol.

Effective methods of providing mechanical force for particle size reduction of benzothiophene, preferably raloxifene hydrochloride include ball milling, media milling, and homogenization, for example, with a Microfluidizer® (Microfluidics Corp.). Ball milling is a low energy milling process that uses milling media, drug, stabilizer, and liquid. The materials are placed in a milling vessel that is rotated at optimal speed such that the media cascades and reduces the drug particle size by impaction. The media used must have a high density as the energy for the particle reduction is provided by gravity and the mass of the attrition media.

Media milling is a high energy milling process. Drug, stabilizer, and liquid are placed in a reservoir and recirculated in a chamber containing media and a rotating shaft/impeller. The rotating shaft agitates the media which subjects the drug to impaction and sheer forces, thereby reducing the drug particle size.

Homogenization is a technique that does not use milling media. Drug, stabilizer, and liquid (or drug and liquid with the stabilizer added after particle size reduction) constitute a process stream propelled into a process zone, which in the Microfluidizer® is called the Interaction Chamber. The product to be treated is inducted into the pump, and then forced out. The priming valve of the Microfluidizer® purges air out of the pump. Once the pump is filled with product, the priming valve is closed and the product is forced through the interaction chamber. The geometry of the interaction chamber produces powerful forces of sheer, impact, and cavitation which are responsible for particle size reduction. Specifically, inside the interaction chamber, the pressurized product is split into two streams and accelerated to extremely high velocities. The formed jets are then directed toward each other and collide in the interaction zone. The resulting product has very fine and uniform particle or droplet size. The Microfluidizer® also provides a heat exchanger to allow cooling of the product. U.S. Pat. No. 5,510,118, which is specifically incorporated by reference, refers to a process using a Microfluidizer® resulting in nanoparticulate particles.

Benzothiophene, preferably raloxifene hydrochloride, can be added to a liquid medium in which it is essentially insoluble to form a premix. The surface stabilizer can be present in the premix, it can be during particle size reduction, or it can be added to the drug dispersion following particle size reduction.

The premix can be used directly by subjecting it to mechanical means to reduce the average benzothiophene, preferably raloxifene hydrochloride, particle size in the dispersion to the desired size, preferably less than about 5 microns. It is preferred that the premix be used directly when a ball mill is used for attrition. Alternatively, benzothiophene, preferably raloxifene hydrochloride, and the surface stabilizer can be dispersed in the liquid media using suitable agitation, e.g., a Cowles type mixer, until a homogeneous dispersion is observed in which there are no large agglomerates visible to the naked eye. It is preferred that the premix be subjected to such a premilling dispersion step when a recirculating media mill is used for attrition.

The mechanical means applied to reduce the benzothiophene, preferably raloxifene hydrochloride, particle size conveniently can take the form of a dispersion mill. Suitable dispersion mills include a ball mill, an attritor mill, a vibratory mill, and media mills such as a sand mill and a bead mill. A media mill is preferred due to the relatively shorter milling time required to provide the desired reduction in particle size. For media milling, the apparent viscosity of the premix is preferably from about 100 to about 1000 centipoise, and for ball milling the apparent viscosity of the premix is preferably from about 1 up to about 100 centipoise. Such ranges tend to afford an optimal balance between efficient particle size reduction and media erosion but are in no way limiting

The attrition time can vary widely and depends primarily upon the particular mechanical means and processing conditions selected. For ball mills, processing times of up to five days or longer may be required. Alternatively, processing times of less than 1 day (residence times of one minute up to several hours) are possible with the use of a high shear media mill.

The benzothiophene, preferably raloxifene hydrochloride, particles must be reduced in size at a temperature which does not significantly degrade benzothiophene, preferably raloxifene hydrochloride. Processing temperatures of less than about 30° to less than about 40° C. are ordinarily preferred. If desired, the processing equipment can be cooled with conventional cooling equipment. Control of the temperature, e.g., by jacketing or immersion of the milling chamber with a cooling liquid, is contemplated. Generally, the method of the invention is conveniently carried out under conditions of ambient temperature and at processing pressures which are safe and effective for the milling process. Ambient processing pressures are typical of ball mills, attritor mills, and vibratory mills.

Grinding Media

The grinding media can comprise particles that are preferably substantially spherical in shape, e.g., beads, consisting essentially of polymeric resin or glass or Zirconium Silicate or other suitable compositions. Alternatively, the grinding media can comprise a core having a coating of a polymeric resin adhered thereon.

In general, suitable polymeric resins are chemically and physically inert, substantially free of metals, solvent, and monomers, and of sufficient hardness and friability to enable them to avoid being chipped or crushed during grinding. Suitable polymeric resins include crosslinked polystyrenes, such as polystyrene crosslinked with divinylbenzene; styrene copolymers; polycarbonates; polyacetals, such as Delrin® (E.I. du Pont de Nemours and Co.); vinyl chloride polymers and copolymers; polyurethanes; polyamides; poly(tetrafluoroethylenes), e.g., Teflon® (E.I. du Pont de Nemours and Co.), and other fluoropolymers; high density polyethylenes; polypropylenes; cellulose ethers and esters such as cellulose acetate; polyhydroxymethacrylate; polyhydroxyethyl acrylate; and silicone-containing polymers such as polysiloxanes and the like. The polymer can be biodegradable. Exemplary biodegradable polymers include poly(lactides), poly(glycolide) copolymers of lactides and glycolide, polyanhydrides, poly(hydroxyethyl methacylate), poly(imino carbonates), poly(N-acylhydroxyproline)esters, poly(N-palmitoyl hydroxyproline) esters, ethylene-vinyl acetate copolymers, poly(orthoesters), poly(caprolactones), and poly(phosphazenes). For biodegradable polymers, contamination from the media itself advantageously can metabolize in vivo into biologically acceptable products that can be eliminated from the body. The polymeric resin can have a density from about 0.8 to about 3.0 g/cm³.

The grinding media preferably ranges in size from about 0.01 to about 3 mm. For fine grinding, the grinding media is preferably from about 0.02 to about 2 mm, and more preferably from about 0.03 to about 1 mm in size.

In one embodiment of the invention, the benzothiophene, preferably raloxifene hydrochloride, particles are made continuously. Such a method comprises continuously introducing benzothiophene, preferably raloxifene hydrochloride, into a milling chamber, contacting the benzothiophene, preferably raloxifene hydrochloride, with grinding media while in the chamber to reduce the benzothiophene, preferably raloxifene hydrochloride, particle size, and continuously removing the nanoparticulate benzothiophene, preferably raloxifene hydrochloride, from the milling chamber.

The grinding media can be separated from the milled nanoparticulate benzothiophene, preferably raloxifene hydrochloride, using conventional separation techniques, in a secondary process such as by simple filtration, sieving through a mesh filter or screen, and the like. Other separation techniques such as centrifugation may also be employed. Alternatively, a screen can be utilized during the milling process to remove the grinding media following completion of particle size reduction.

### F. Method of Treatment

The present invention is also directed to methods treatment or prevention using the nanoparticulate benzothiophene or a salt thereof, preferably raloxifene hydrochloride, compositions of the invention for conditions such as osteoporosis or related conditions, such as Paget's disease, carcinomas of the breast and lymph glands, and the like.

For example, the nanoparticulate composition may be used to treat breast cancer and other tumors of the breast and lymph nodular tissues. The compositions may also be used to treat or prevent osteoporosis or related conditions. The composition may further comprise at least one surface stabilizer adsorbed to or associated with the surface of the benzothiophene nanoparticles. In one embodiment, the nanoparticulate benzothiophene is a nanoparticulate raloxifene hydrochloride.

Such treatment comprises administering to the subject the nanoparticulate benzothiophene, preferably raloxifene hydrochloride, formulation of the invention. As used herein, the term “subject” is used to mean an animal, preferably a mammal, including a human or non-human. The terms patient and subject may be used interchangeably.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents, or vehicles including water, ethanol, polyols (propyleneglycol, polyethylene-glycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions, and by the use of surfactants.

The nanoparticulate compositions may also contain adjuvants such as preserving, wetting, emulsifying, and dispensing agents. Prevention of the growth of microorganisms can be ensured by various antibacterial and antifungal agents, such as parabens, chlorobutanol, phenol, sorbic acid, and the like. It may also be desirable to include isotonic agents, such as sugars, sodium chloride, and the like. Prolonged absorption of the injectable pharmaceutical form can be brought about by the use of agents delaying absorption, such as aluminum monostearate and gelatin.

One of ordinary skill will appreciate that effective amounts of benzothiophene, preferably raloxifene hydrochloride, can be determined empirically and can be employed in pure form or, where such forms exist, in pharmaceutically acceptable salt, ester, or prodrug form. Actual dosage levels of benzothiophene, preferably raloxifene hydrochloride, in the nanoparticulate compositions of the invention may be varied to obtain an amount of benzothiophene, preferably raloxifene hydrochloride, that is effective to obtain a desired therapeutic response for a particular composition and method of administration. The selected dosage level therefore depends upon the desired therapeutic effect, the route of administration, the potency of the administered benzothiophene, preferably raloxifene hydrochloride, the desired duration of treatment, and other factors.

Dosage unit compositions may contain such amounts of such submultiples thereof as may be used to make up the daily or other suitable dosing period (e.g., such as every other day, weekly, bi-weekly, monthly, etc.) It will be understood, however, that the specific dose level for any particular patient will depend upon a variety of factors: the type and degree of the cellular or physiological response to be achieved; activity of the specific agent or composition employed; the specific agents or composition employed; the age, body weight, general health, sex, and diet of the patient; the time of administration, route of administration, and rate of excretion of the agent; the duration of the treatment; drugs used in combination or coincidental with the specific agent; and like factors well known in the medical arts.

The following examples are given to illustrate the present invention. It should be understood, however, that the spirit and scope of the invention is not to be limited to the specific conditions or details described in these examples but should only be limited by the scope of the claims that follow. All references identified herein, including U.S. patents, are hereby expressly incorporated by reference.

### EXAMPLE 1

The purpose of this example was to prepare a nanoparticulate formulation of raloxifene hydrochloride.

An aqueous dispersion of 5% (w/w) raloxifene hydrochloride (Manufacturer: Aarti Drugs Ltd; Supplier: Camida Ltd.; Batch Number: RAL/503009), combined with 2% (w/w) Pharmacoat® 603 (hydroxypropyl methylcellulose), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 211 nm, with a D50 of 204 nm, a D90 of 271 nm, and a D95 of 296 nm.

The particle size was also measured in media representative of biological conditions (i.e., “biorelevant media”). Biorelevant aqueous media can be any aqueous media that exhibit the desired ionic strength and pH, which form the basis for the biorelevance of the media. The desired pH and ionic strength are those that are representative of physiological conditions found in the human body. Such biorelevant aqueous media can be, for example, aqueous electrolyte solutions or aqueous solutions of any salt, acid, or base, or a combination thereof, which exhibit the desired pH and ionic strength.

Biorelevant pH is well known in the art. For example, in the stomach, the pH ranges from slightly less than 2 (but typically greater than 1) up to 4 or 5. In the small intestine the pH can range from 4 to 6, and in the colon it can range from 6 to 8. Biorelevant ionic strength is also well known in the art. Fasted state gastric fluid has an ionic strength of about 0.1M while fasted state intestinal fluid has an ionic strength of about 0.14. See e.g., Lindahl et al., “Characterization of Fluids from the Stomach and Proximal Jejunum in Men and Women,” Pharm. Res., 14 (4): 497-502 (1997).

It is believed that the pH and ionic strength of the test solution is more critical than the specific chemical content. Accordingly, appropriate pH and ionic strength values can be obtained through numerous combinations of strong acids, strong bases, salts, single or multiple conjugate acid-base pairs (i.e., weak acids and corresponding salts of that acid), monoprotic and polyprotic electrolytes, etc.

Representative electrolyte solutions can be, but are not limited to, HCl solutions, ranging in concentration from about 0.001 to about 0.1 M, and NaCl solutions, ranging in concentration from about 0.001 to about 0.1 M, and mixtures thereof. For example, electrolyte solutions can be, but are not limited to, about 0.1 M HCl or less, about 0.01 M HCl or less, about 0.001 M HCl or less, about 0.1 M NaCl or less, about 0.01 M NaCl or less, about 0.001 M NaCl or less, and mixtures thereof. Of these electrolyte solutions, 0.01 M HCl and/or 0.1 M NaCl, are most representative of fasted human physiological conditions, owing to the pH and ionic strength conditions of the proximal gastrointestinal tract.

Electrolyte concentrations of 0.001 M HCl, 0.01 M HCl, and 0.1 M HCl correspond to pH 3, pH 2, and pH 1, respectively. Thus, a 0.01 M HCl solution simulates typical acidic conditions found in the stomach. A solution of 0.1 M NaCl provides a reasonable approximation of the ionic strength conditions found throughout the body, including the gastrointestinal fluids, although concentrations higher than 0.1 M may be employed to simulate fed conditions within the human GI tract.

The particle size in various biorelevant media is shown in Table 1, below.

TABLE 1

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 178 | 172 | 238 | 258 |
| 0.1 M NaCl | 179 | 173 | 238 | 258 |
| 0.01 N HCl | 198 | 192 | 256 | 283 |
| 0.01 N HCl | 203 | 197 | 260 | 287 |

The stability of the milled raloxifene hydrochloride was measured over a seven day period under various temperature conditions. The results of the stability test are show below in Table 2.

TABLE 2

| Storage | Storage | | | | |
|---|---|---|---|---|---|
| condition time | Condition | Mean/nm | D50/nm | D90/nm | D95/nm |
| Time = 0 Days | Ambient | 207 | 200 | 264 | 292 |
| Time = 0 Days | Ambient | 284 | 290 | 430 | 473 |
| Time = 7 Days | 5° C. | 216 | 209 | 280 | 308 |
| Time = 7 Days | 5° C. | 224 | 216 | 291 | 325 |
| Time = 7 Days | 25° C. | 218 | 210 | 283 | 314 |
| Time = 7 Days | 25° C. | 220 | 211 | 288 | 320 |
| Time = 7 Days | 40° C. | 230 | 221 | 296 | 331 |
| Time = 7 Days | 40° C. | 235 | 225 | 307 | 338 |

### EXAMPLE 2

The purpose of this example was to prepare a nanoparticulate formulation of raloxifene hydrochloride.

An aqueous dispersion of 5% (w/w) raloxifene hydrochloride (Camida Ltd.), combined with 2% (w/w) HPC-SL (hydroxypropyl cellulose—super low viscosity), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 198 nm, with a D50 of 193 nm, a D90 of 252 nm, and a D95 of 277 nm.

The particle size measured in various biorelevant media is shown in Table 3, below.

TABLE 3

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 184 | 179 | 243 | 264 |
| 0.1 M NaCl | 184 | 179 | 243 | 262 |
| 0.01 N HCl | 192 | 187 | 250 | 273 |
| 0.01 N HCl | 195 | 189 | 251 | 275 |

The stability of the milled raloxifene hydrochloride was measured over a seven day period under various temperature conditions. The results of the stability test are show below in Table 4.

TABLE 4

| Storage | | Mean/ | | D90/ | D95/ |
|---|---|---|---|---|---|
| condition time | Storage Condition | nm | D50/nm | nm | nm |
| Time = 0 Days | Ambient | 204 | 198 | 258 | 286 |
| Time = 0 Days | Ambient | 226 | 224 | 301 | 328 |
| Time = 7 Days | 5° C. | 201 | 195 | 257 | 284 |
| Time = 7 Days | 5° C. | 195 | 189 | 252 | 278 |
| Time = 7 Days | 25° C. | 206 | 200 | 263 | 290 |
| Time = 7 Days | 25° C. | 202 | 196 | 260 | 287 |
| Time = 7 Days | 40° C. | 216 | 210 | 280 | 306 |
| Time = 7 Days | 40° C. | 218 | 212 | 282 | 308 |

### EXAMPLE 3

The purpose of this example was to prepare a nanoparticulate formulation of raloxifene hydrochloride.

An aqueous dispersion of 5% (w/w) raloxifene hydrochloride (Camida Ltd.), combined with 2% (w/w) Plasdone S630 (copovidone K25-34), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 225 nm, with a D50 of 212 nm, a D90 of 298 nm, and a D95 of 344 nm.

The particle size measured in various biorelevant media is shown in Table 5, below.

TABLE 5

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 174 | 167 | 240 | 267 |
| 0.1 M NaCl | 176 | 170 | 242 | 268 |
| 0.01 N HCl | 186 | 179 | 247 | 274 |
| 0.01 N HCl | 188 | 182 | 247 | 270 |

The stability of the milled raloxifene hydrochloride was measured over a seven day period under various temperature conditions. The results of the stability test are show below in Table 6.

TABLE 6

| Storage | | Mean/ | D50/ | D90/ | D95/ |
|---|---|---|---|---|---|
| condition time | Storage Condition | nm | nm | nm | nm |
| Time = 0 Days | Ambient | 202 | 194 | 257 | 288 |
| Time = 0 Days | Ambient | 309 | 314 | 472 | 516 |
| Time = 7 Days | 5° C. | 207 | 200 | 270 | 297 |
| Time = 7 Days | 5° C. | 236 | 222 | 318 | 364 |
| Time = 7 Days | 25° C. | 212 | 204 | 277 | 306 |
| Time = 7 Days | 25° C. | 227 | 217 | 297 | 335 |
| Time = 7 Days | 40° C. | 203 | 192 | 285 | 326 |
| Time = 7 Days | 40° C. | 216 | 202 | 308 | 354 |

### EXAMPLE 4

The purpose of this example was to prepare a nanoparticulate formulation of raloxifene hydrochloride.

An aqueous dispersion of 5% (w/w) raloxifene hydrochloride (Camida Ltd.), combined with 2% (w/w) Plasdone K29/32 (povidone K29-32), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 186 nm, with a D50 of 180 nm, a D90 of 242 nm, and a D95 of 263 nm.

The particle size measured in various biorelevant media is shown in Table 7, below.

TABLE 7

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 177 | 169 | 247 | 278 |
| 0.1 M NaCl | 173 | 166 | 239 | 265 |
| 0.01 N HCl | 189 | 181 | 254 | 285 |
| 0.01 N HCl | 179 | 173 | 237 | 257 |

The stability of the milled raloxifene hydrochloride was measured over a seven day period under various temperature conditions. The results of the stability test are show below in Table 8.

TABLE 8

| Storage | | Mean/ | D50/ | D90/ | D95/ |
|---|---|---|---|---|---|
| condition time | Storage Condition | nm | nm | nm | nm |
| Time = 0 Days | Ambient | 192 | 186 | 247 | 271 |
| Time = 0 Days | Ambient | 221 | 204 | 380 | 425 |
| Time = 7 Days | 5° C. | 193 | 187 | 252 | 278 |
| Time = 7 Days | 5° C. | 198 | 191 | 256 | 284 |
| Time = 7 Days | 25° C. | 188 | 182 | 247 | 270 |
| Time = 7 Days | 25° C. | 193 | 187 | 252 | 279 |
| Time = 7 Days | 40° C. | 198 | 191 | 256 | 284 |
| Time = 7 Days | 40° C. | 202 | 196 | 263 | 290 |

### EXAMPLE 5

The purpose of this example was to prepare a nanoparticulate formulation of raloxifene hydrochloride.

An aqueous dispersion of 5% (w/w) raloxifene hydrochloride (Camida Ltd.), combined with 1.5% (w/w) Tween 80 (polyoxyethylene sorbitan fatty acid ester 80), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident. However, there were some slightly larger crystal, possibly either “un-milled” drug or signs of crystal growth.

Following milling, the particle size of the milled raloxifene hydrochloride particles was measured, in deionized distilled water, using a Horiba LA 910 particle size analyzer. The mean milled raloxifene hydrochloride particle size was 513 nm, with a D50 of 451 nm, a D90 of 941 nm, and a D95 of 1134 nm. The sample was measured two additional times in the distilled water, resulting in mean raloxifene hydrochloride particle sizes of 328 and 1671 nm, D50 of 109 and 1115 nm, D90 of 819 and 3943 nm, and a D95 of 1047 and 4983 nm.

The particle size measured in various biorelevant media is shown in Table 9, below.

TABLE 9

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 231 | 222 | 300 | 334 |
| 0.1 M NaCl | 233 | 224 | 303 | 335 |
| 0.01 N HCl | 307 | 297 | 424 | 470 |
| 0.01 N HCl | 321 | 309 | 447 | 502 |

The stability of the milled raloxifene hydrochloride was measured over a seven day period under various temperature conditions. The results of the stability test are show below in Table 10.

TABLE 10

| Storage | | Mean/ | | D90/ | D95/ |
|---|---|---|---|---|---|
| condition time | Storage Condition | nm | D50/nm | nm | nm |
| Time = 0 Days | Ambient | 683 | 426 | 1568 | 2172 |
| Time = 0 Days | Ambient | 751 | 568 | 1437 | 1836 |
| Time = 7 Days | 5° C. | 578 | 424 | 1156 | 1532 |
| Time = 7 Days | 5° C. | 631 | 464 | 1247 | 1630 |
| Time = 7 Days | 25° C. | 599 | 458 | 1155 | 1478 |
| Time = 7 Days | 25° C. | 628 | 490 | 1198 | 1492 |
| Time = 7 Days | 40° C. | — | — | — | — |
| Time = 7 Days | 40° C. | — | — | — | — |

### EXAMPLE 6

The purpose of this example was to prepare a nanoparticulate formulation of raloxifene hydrochloride.

An aqueous dispersion of 5% (w/w) raloxifene hydrochloride (Camida Ltd.), combined with 1.25% (w/w) Plasdone S630 (copovidone K25-34) and 0.05% (w/w) sodium lauryl sulfate, was milled in a 10 ml chamber of a NanoMill® 0.01 (NanoMill Systems, King of Prussia, Pa.; see e.g., U.S. Pat. No. 6,431,478), along with 500 micron PolyMill® 500 attrition media (Dow Chemical) (89% media load). The mixture was milled at a speed of 3500 rpms for 60 min., and a second sample was milled for 90 mm.

Microscopy of the milled sample, using a Lecia DM5000B and Lecia CTR 5000 light source (Laboratory Instruments and Supplies Ltd., Ashbourne Co., Meath, Ireland), showed brownian motion in part, but a large number of flocculated particles was also observed.

Following milling, the particle size of the milled raloxifene hydrochloride particles was measured, in deionized distilled water, using a Horiba LA 910 particle size analyzer. The mean milled raloxifene hydrochloride particle size was 178 nm, with a D50 of 132 nm, a D90 of 347 nm, and a D95 of 412 nm, and in a second measurement the sample had a mean particle size of 617 nm, a D50 of 277 nm, a D90 of 1905, and a D95 of 2692. Following 90 min. of milling, the mean milled raloxifene hydrochloride particle size was 867 nm, with a D50 of 380 nm, a D90 of 2342 nm, and a D95 of 2982 nm, and in a second measurement the sample had a mean particle size of 1885 nm, a D50 of 877 nm, a D90 of 4770 nm, and a D95 of 5863 nm.

The particle size measured in various biorelevant media is shown in Table 11, below.

TABLE 11

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 103 | 99 | 157 | 177 |
| 0.1 M NaCl | 104 | 100 | 159 | 179 |
| 0.01 N HCl | 112 | 108 | 167 | 189 |
| 0.01 N HCl | 139 | 139 | 186 | 202 |

The stability of the milled raloxifene hydrochloride was measured over a seven day period under various temperature conditions. The results of the stability test are show below in Table 12.

TABLE 12

| Storage | | Mean/ | D50/ | D90/ | D95/ |
|---|---|---|---|---|---|
| condition time | Storage Condition | nm | nm | nm | nm |
| Time = 0 Days | Ambient | 135 | 112 | 233 | 288 |
| Time = 0 Days | Ambient | 177 | 128 | 280 | 382 |
| Time = 7 Days | 5° C. | 155 | 151 | 211 | 232 |
| Time = 7 Days | 5° C. | 298 | 181 | 313 | 832 |
| Time = 7 Days | 25° C. | 161 | 157 | 215 | 235 |
| Time = 7 Days | 25° C. | 179 | 173 | 240 | 263 |
| Time = 7 Days | 40° C. | 182 | 177 | 239 | 258 |
| Time = 7 Days | 40° C. | 199 | 194 | 257 | 285 |

### EXAMPLE 7

The purpose of this example was to prepare a nanoparticulate formulation of raloxifene hydrochloride.

An aqueous dispersion of 5% (w/w) raloxifene hydrochloride (Camida Ltd.), combined with 1.25% (w/w) Plasdone K29/32 (povidone K29/32) and 0.05% (w/w) sodium lauryl sulfate, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 182 nm, with a D50 of 176 nm, a D90 of 238 nm, and a D95 of 258 nm. In a second measurement in distilled water, the mean raloxifene hydrochloride particle size was 250 nm, with a D50 of 244 nm, a D90 of 337 nm, and a D95 of 373 nm.

The particle size measured in various biorelevant media is shown in Table 13, below.

TABLE 13

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 149 | 144 | 207 | 228 |
| 0.1 M NaCl | 149 | 144 | 205 | 225 |
| 0.01 N HCl | 163 | 158 | 218 | 241 |
| 0.01 N HCl | 165 | 161 | 219 | 240 |

The stability of the milled raloxifene hydrochloride was measured over a seven day period under various temperature conditions. The results of the stability test are show below in Table 14.

TABLE 14

| Storage | | Mean/ | D50/ | D90/ | D95/ |
|---|---|---|---|---|---|
| condition time | Storage Condition | nm | nm | nm | nm |
| Time = 0 Days | Ambient | 180 | 174 | 233 | 254 |
| Time = 0 Days | Ambient | 201 | 169 | 364 | 411 |
| Time = 7 Days | 5° C. | 184 | 178 | 241 | 262 |
| Time = 7 Days | 5° C. | 195 | 189 | 256 | 285 |
| Time = 7 Days | 25° C. | 188 | 182 | 247 | 270 |
| Time = 7 Days | 25° C. | 195 | 188 | 254 | 282 |
| Time = 7 Days | 40° C. | 209 | 203 | 271 | 294 |
| Time = 7 Days | 40° C. | 217 | 210 | 282 | 310 |

### EXAMPLE 8

The purpose of this example was to prepare a nanoparticulate formulation of raloxifene hydrochloride.

An aqueous dispersion of 5% (w/w) raloxifene hydrochloride (Camida Ltd.), combined with 1.25% (w/w) HPC-SL (hydroxypropyl cellulose—super low viscosity) and 0.05% (w/w) docusate sodium, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 192 nm, with a D50 of 186 nm, a D90 of 248 nm, and a D95 of 272 nm. In a second measurement in distilled water, the mean raloxifene hydrochloride particle size was 193 nm, with a D50 of 187 nm, a D90 of 250 nm, and a D95 of 274 nm.

The particle size measured in various biorelevant media is shown in Table 15, below.

TABLE 15

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 185 | 180 | 247 | 271 |
| 0.1 M NaCl | 183 | 178 | 243 | 265 |
| 0.01 N HCl | 200 | 194 | 257 | 285 |
| 0.01 N HCl | 206 | 200 | 265 | 290 |

The stability of the milled raloxifene hydrochloride was measured over a seven day period under various temperature conditions. The results of the stability test are show below in Table 16.

TABLE 16

| Storage | | Mean/ | D50/ | D90/ | D95/ |
|---|---|---|---|---|---|
| condition time | Storage Condition | nm | nm | nm | nm |
| Time = 0 Days | Ambient | 200 | 194 | 255 | 283 |
| Time = 0 Days | Ambient | 201 | 195 | 256 | 283 |
| Time = 7 Days | 5° C. | 207 | 201 | 267 | 292 |
| Time = 7 Days | 5° C. | 207 | 202 | 267 | 292 |
| Time = 7 Days | 25° C. | 213 | 206 | 276 | 301 |
| Time = 7 Days | 25° C. | 213 | 206 | 276 | 301 |
| Time = 7 Days | 40° C. | 207 | 195 | 290 | 330 |
| Time = 7 Days | 40° C. | 212 | 199 | 299 | 340 |

### EXAMPLE 9

The purpose of this example was to prepare a nanoparticulate formulation of raloxifene hydrochloride.

An aqueous dispersion of 5% (w/w) raloxifene hydrochloride (Camida Ltd.), combined with 1.25% (w/w) Pharmacoat 603 (hydroxypropyl cellulose) and 0.05% (w/w) docusate sodium, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed brownian motion in part, but also demonstrated a large number of flocculated particles.

Following milling, the particle size of the milled raloxifene hydrochloride particles was measured, in deionized distilled water, using a Horiba LA 910 particle size analyzer. The mean milled raloxifene hydrochloride particle size was 213 nm, with a D50 of 205 nm, a D90 of 275 nm, and a D95 of 301 nm. In a second measurement in distilled water, the mean raloxifene hydrochloride particle size was 216 nm, with a D50 of 209 nm, a D90 of 280 nm, and a D95 of 309 nm.

The particle size measured in various biorelevant media is shown in Table 17, below.

TABLE 17

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 182 | 176 | 243 | 266 |
| 0.1 M NaCl | 183 | 177 | 243 | 266 |
| 0.01 N HCl | 201 | 194 | 258 | 286 |
| 0.01 N HCl | 207 | 201 | 267 | 292 |

The stability of the milled raloxifene hydrochloride was measured over a seven day period under various temperature conditions. The results of the stability test are show below in Table 18.

TABLE 18

| Storage | | Mean/ | | D90/ | |
|---|---|---|---|---|---|
| condition time | Storage Condition | nm | D50/nm | nm | D95/nm |
| Time = 0 Days | Ambient | 213 | 205 | 275 | 301 |
| Time = 0 Days | Ambient | 216 | 209 | 280 | 309 |
| Time = 7 Days | 5° C. | 215 | 208 | 279 | 306 |
| Time = 7 Days | 5° C. | 218 | 210 | 282 | 311 |
| Time = 7 Days | 25° C. | 225 | 216 | 292 | 325 |
| Time = 7 Days | 25° C. | 227 | 218 | 295 | 330 |
| Time = 7 Days | 40° C. | 213 | 201 | 302 | 344 |
| Time = 7 Days | 40° C. | 221 | 208 | 317 | 362 |

### EXAMPLE 10

The purpose of this example was to prepare a nanoparticulate formulation of raloxifene hydrochloride.

An aqueous dispersion of 5% (w/w) raloxifene hydrochloride (Camida Ltd.), combined with 0.1% (w/w) docusate sodium, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation. However, a few larger, possible “un-milled” drug or recrystallizastion was observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 206 nm, with a D50 of 199 nm, a D90 of 267 nm, and a D95 of 293 nm. In a second measurement in distilled water, the mean raloxifene hydrochloride particle size was 228 nm, with a D50 of 218 nm, a D90 of 295 nm, and a D95 of 332 nm.

The stability of the milled raloxifene hydrochloride was measured over a seven day period under various temperature conditions. The results of the stability test are show below in Table 19.

TABLE 19

| Storage | | Mean/ | D50/ | D90/ | D95/ |
|---|---|---|---|---|---|
| condition time | Storage Condition | nm | nm | nm | nm |
| Time = 0 Days | Ambient | 206 | 199 | 267 | 293 |
| Time = 0 Days | Ambient | 228 | 218 | 295 | 332 |
| Time = 7 Days | 5° C. | 226 | 217 | 293 | 328 |
| Time = 7 Days | 5° C. | 209 | 197 | 292 | 334 |
| Time = 7 Days | 25° C. | 215 | 202 | 306 | 352 |
| Time = 7 Days | 25° C. | 284 | 273 | 387 | 435 |
| Time = 7 Days | 40° C. | 220 | 209 | 312 | 352 |
| Time = 7 Days | 40° C. | — | — | — | — |

### EXAMPLE 11

The purpose of this example was to prepare a nanoparticulate formulation of raloxifene hydrochloride.

An aqueous dispersion of 5% (w/w) raloxifene hydrochloride (Camida Ltd.), combined with 0.1% (w/w) sodium lauryl sulfate, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident. There were signs of flocculation and also signs of “un-milled” drug crystals. The sample, however, appears acceptable.

Following milling, the particle size of the milled raloxifene hydrochloride particles was measured, in deionized distilled water, using a Horiba LA 910 particle size analyzer. The mean milled raloxifene hydrochloride particle size was 186 nm, with a D50 of 180 nm, a D90 of 242 nm, and a D95 of 263 nm. In a second measurement in distilled water, the mean raloxifene hydrochloride particle size was 204 nm, with a D50 of 168 nm, a D90 of 374 nm, and a D95 of 426 nm.

The particle size measured in various biorelevant media is shown in Table 20, below.

TABLE 20

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 463 | 185 | 300 | 425 |
| 0.1 M NaCl | 245 | 233 | 327 | 369 |
| 0.01 N HCl | 197 | 192 | 253 | 277 |
| 0.01 N HCl | 201 | 196 | 256 | 282 |

The stability of the milled raloxifene hydrochloride was measured over a seven day period under various temperature conditions. The results of the stability test are show below in Table 21.

TABLE 21

| Storage | | Mean/ | D50/ | D90/ | D95/ |
|---|---|---|---|---|---|
| condition time | Storage Condition | nm | nm | nm | nm |
| Time = 0 Days | Ambient | 213 | 206 | 271 | 295 |
| Time = 0 Days | Ambient | 211 | 198 | 295 | 339 |
| Time = 4 Days | 5° C. | 213 | 207 | 277 | 301 |
| Time = 4 Days | 5° C. | 220 | 212 | 287 | 318 |
| Time = 4 Days | 25° C. | 225 | 217 | 289 | 320 |
| Time = 4 Days | 25° C. | 225 | 216 | 290 | 322 |
| Time = 4 Days | 40° C. | 316 | 304 | 438 | 491 |
| Time = 4 Days | 40° C. | 339 | 321 | 486 | 553 |

### EXAMPLE 12

The purpose of this example was to prepare a nanoparticulate formulation of raloxifene hydrochloride.

An aqueous dispersion of 5% (w/w) raloxifene hydrochloride (Camida Ltd.), combined with 1.5% (w/w) Pluronic F108 (poloxamer 308), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 215 nm, with a D50 of 122 nm, a D90 of 475 nm, and a D95 of 648 nm. In a second measurement in distilled water, the mean raloxifene hydrochloride particle size was 185 nm, with a D50 of 116 nm, a D90 of 395 nm, and a D95 of 473 nm.

The particle size measured in various biorelevant media is shown in Table 22, below.

TABLE 22

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 210 | 204 | 273 | 295 |
| 0.1 M NaCl | 212 | 206 | 275 | 297 |
| 0.01 N HCl | 196 | 184 | 276 | 317 |
| 0.01 N HCl | 269 | 263 | 363 | 394 |

The stability of the milled raloxifene hydrochloride was measured over a seven day period under various temperature conditions. The results of the stability test are show below in Table 23.

TABLE 23

| Storage | | Mean/ | D50/ | D90/ | D95/ |
|---|---|---|---|---|---|
| condition time | Storage Condition | nm | nm | nm | nm |
| Time = 0 Days | Ambient | 374 | 326 | 551 | 728 |
| Time = 0 Days | Ambient | 510 | 386 | 895 | 1474 |
| Time = 4 Days | 5° C. | 546 | 341 | 1082 | 2008 |
| Time = 4 Days | 5° C. | 642 | 389 | 1494 | 2279 |
| Time = 4 Days | 25° C. | 2378 | 826 | 6793 | 8639 |
| Time = 4 Days | 25° C. | 3021 | 1523 | 7876 | 9866 |
| Time = 4 Days | 40° C. | 3631 | 2245 | 8729 | 10826 |
| Time = 4 Days | 40° C. | 4019 | 2817 | 9179 | 11283 |

### EXAMPLE 13

The purpose of this example was to prepare a nanoparticulate formulation of raloxifene hydrochloride.

An aqueous dispersion of 5% (w/w) raloxifene hydrochloride (Camida Ltd.), combined with 1.25% (w/w) Lutrol F68 (polyoxamer 188) and 0.05% (w/w) docusate sodium, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 283 nm, with a D50 of 289 nm, a D90 of 436 nm, and a D95 of 483 nm. In a second measurement in distilled water, the mean raloxifene hydrochloride particle size was 279 nm, with a D50 of 270 nm, a D90 of 369 nm, and a D95 of 407 nm.

The particle size measured in various biorelevant media is shown in Table 24, below.

TABLE 24

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 233 | 223 | 306 | 338 |
| 0.1 M NaCl | 218 | 205 | 310 | 357 |
| 0.01 N HCl | 202 | 191 | 284 | 324 |
| 0.01 N HCl | 260 | 253 | 348 | 381 |

The stability of the milled raloxifene hydrochloride was measured over a seven day period under various temperature conditions. The results of the stability test are show below in Table 25.

TABLE 25

| Storage | | Mean/ | D50/ | D90/ | D95/ |
|---|---|---|---|---|---|
| condition time | Storage Condition | nm | nm | nm | nm |
| Time = 4 Days | Ambient | 321 | 303 | 461 | 531 |
| Time = 0 Days | Ambient | 281 | 273 | 381 | 423 |
| Time = 4 Days | 5° C. | 276 | 265 | 378 | 426 |
| Time = 4 Days | 5° C. | 278 | 267 | 383 | 433 |
| Time = 4 Days | 25° C. | 508 | 299 | 574 | 1763 |
| Time = 4 Days | 25° C. | 584 | 313 | 1173 | 2487 |
| Time = 4 Days | 40° C. | 1232 | 332 | 4150 | 7902 |
| Time = 4 Days | 40° C. | 1435 | 351 | 5359 | 8299 |

### EXAMPLE 14

The purpose of this example was to prepare a nanoparticulate formulation of raloxifene hydrochloride.

An aqueous dispersion of 5% (w/w) raloxifene hydrochloride (Camida Ltd.), combined with 1.25% (w/w) Plasdone C-15 (povidone K15.5-17.5) and 0.05% (w/w) deoxycholic acid, sodium salt, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 169 nm, with a D50 of 164 nm, a D90 of 220 nm, and a D95 of 242 nm. In a second measurement in distilled water, the mean raloxifene hydrochloride particle size was 179 nm, with a D50 of 171 nm, a D90 of 271 nm, and a D95 of 298 nm.

The particle size measured in various biorelevant media is shown in Table 26, below.

TABLE 26

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 146 | 141 | 199 | 221 |
| 0.1 M NaCl | 147 | 143 | 200 | 221 |
| 0.01 N HCl | 152 | 150 | 203 | 222 |
| 0.01 N HCl | 158 | 155 | 209 | 225 |

The stability of the milled raloxifene hydrochloride was measured over a seven day period under various temperature conditions. The results of the stability test are show below in Table 27.

TABLE 27

| Storage | | Mean/ | D50/ | D90/ | D95/ |
|---|---|---|---|---|---|
| condition time | Storage Condition | nm | nm | nm | nm |
| Time = 0 Days | Ambient | 186 | 180 | 238 | 258 |
| Time = 0 Days | Ambient | 203 | 198 | 306 | 336 |
| Time = 4 Days | 5° C. | 165 | 160 | 219 | 242 |
| Time = 4 Days | 5° C. | 168 | 163 | 222 | 246 |
| Time = 4 Days | 25° C. | 187 | 182 | 244 | 266 |
| Time = 4 Days | 25° C. | 187 | 151 | 343 | 388 |
| Time = 4 Days | 40° C. | 195 | 189 | 253 | 279 |
| Time = 4 Days | 40° C. | 197 | 191 | 256 | 283 |

### EXAMPLE 15

The purpose of this example was to prepare a nanoparticulate formulation of raloxifene hydrochloride.

An aqueous dispersion of 5% (w/w) raloxifene hydrochloride (Camida Ltd.), combined with 1.5% (w/w) Lutrol F 127 (poloxamer 407), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 209 nm, with a D50 of 158 nm, a D90 of 396 nm, and a D95 of 454 nm. In a second measurement in distilled water, the mean raloxifene hydrochloride particle size was 197 nm, with a D50 of 125 nm, a D90 of 410 nm, and a D95 of 479 nm.

The particle size measured in various biorelevant media is shown in Table 28, below.

TABLE 28

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 197 | 191 | 257 | 285 |
| 0.1 M NaCl | 200 | 194 | 261 | 288 |
| 0.01 N HCl | 267 | 261 | 359 | 387 |
| 0.01 N HCl | 278 | 270 | 377 | 417 |

The stability of the milled raloxifene hydrochloride was measured over a seven day period under various temperature conditions. The results of the stability test are show below in Table 29.

TABLE 29

| Storage | | Mean/ | D50/ | D90/ | D95/ |
|---|---|---|---|---|---|
| condition time | Storage Condition | nm | nm | nm | nm |
| Time = 0 Days | Ambient | 228 | 160 | 369 | 756 |
| Time = 0 Days | Ambient | 225 | 126 | 449 | 688 |
| Time = 4 Days | 5° C. | 306 | 289 | 433 | 498 |
| Time = 4 Days | 5° C. | 473 | 331 | 775 | 1519 |
| Time = 4 Days | 5° C. (Repeat) | 394 | 352 | 617 | 746 |
| Time = 4 Days | 5° C. (Repeat) | 463 | 429 | 697 | 816 |
| Time = 4 Days | 25° C. | 886 | 361 | 2764 | 4102 |
| Time = 4 Days | 25° C. | — | — | — | — |
| Time = 4 Days | 40° C. | 1084 | 520 | 2923 | 4221 |
| Time = 4 Days | 40° C. | 1276 | 580 | 3512 | 4888 |

### EXAMPLE 16

The purpose of this example was to prepare a nanoparticulate formulation of raloxifene hydrochloride.

An aqueous dispersion of 5% (w/w) raloxifene hydrochloride (Camida Ltd.), combined with 1.0% (w/w) Pluronic F108 (poloxamer 308) and 1.0% (w/w) Tween 80 (polyoxyethylene sorbitan fatty acid ester 80), was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation. Throughout the sample larger, possibly “un-milled” drug crystals or crystal growth, however, was observed. Nonetheless, the sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 180 nm, with a D50 of 88 nm, a D90 of 562 nm, and a D95 of 685 nm. In a second measurement in distilled water, the mean raloxifene hydrochloride particle size was 186 nm, with a D50 of 88 nm, a D90 of 605 nm, and a D95 of 762 nm.

The particle size measured in various biorelevant media is shown in Table 30, below.

TABLE 30

| Biorelevant | Mean Particle | D50 Particle | D90 Particle | D95 Particle |
|---|---|---|---|---|
| Media | Size (nm) | Size (nm) | Size (nm) | Size (nm) |
| 0.1 M NaCl | 208 | 202 | 271 | 294 |
| 0.1 M NaCl | 211 | 205 | 275 | 298 |
| 0.01 N HCl | 263 | 257 | 350 | 382 |
| 0.01 N HCl | 279 | 272 | 377 | 417 |

The stability of the milled raloxifene hydrochloride was measured over a seven day period under various temperature conditions. The results of the stability test are show below in Table 31.

TABLE 31

| Storage | | Mean/ | D50/ | D90/ | D95/ |
|---|---|---|---|---|---|
| condition time | Storage Condition | nm | nm | nm | nm |
| Time = 0 Days | Ambient | 2077 | 1584 | 4636 | 5406 |
| Time = 0 Days | Ambient | 2019 | 1594 | 4450 | 5127 |
| Time = 4 Days | 5° C. | 497 | 457 | 761 | 890 |
| Time = 4 Days | 5° C. | 458 | 422 | 705 | 825 |
| Time = 4 Days | 5° C. | 480 | 438 | 748 | 876 |
| Time = 4 Days | 25° C. | 431 | 397 | 657 | 764 |
| Time = 4 Days | 25° C. | 453 | 415 | 702 | 827 |
| Time = 4 Days | 40° C. | 566 | 486 | 968 | 1174 |
| Time = 4 Days | 40° C. | 612 | 524 | 1051 | 1265 |

### EXAMPLE 17

The purpose of this example was to prepare a nanoparticulate formulation of raloxifene hydrochloride.

An aqueous dispersion of 5% (w/w) raloxifene hydrochloride (Camida Ltd.), combined with 1.25% (w/w) Plasdone K-17 (povidone K17) and 0.05% (w/w) benzalkonium chloride, was milled in a 10 ml chamber of a NanoMill® 0.01 (NanoMill Systems, King of Prussia, Pa.; see e.g., U.S. Pat. No. 6,431,478), along with 500 micron PolyMill® attrition media (Dow Chemical) (89% media load). The mixture was milled at a speed of 2500 rpms for 60 min.

Microscopy of the milled sample, using a Lecia DM5000B and Lecia CTR 5000 light source (Laboratory Instruments and Supplies Ltd., Ashbourne Co., Meath, Ireland), showed well dispersed discrete particles. Brownian motion was also clearly evident with no signs of flocculation or crystal growth. Larger “un-milled” drug was not observed. The sample appeared acceptable.

Following milling, the particle size of the milled raloxifene hydrochloride particles was measured, in deionized distilled water, using a Horiba LA 910 particle size analyzer. The mean milled raloxifene hydrochloride particle size was 195 nm, with a D50 of 187 nm, a D90 of 254 nm, and a D95 of 283 nm. In a second measurement in distilled water, the mean raloxifene hydrochloride particle size was 213 nm, with a D50 of 190 nm, a D90 of 375 nm, and a D95 of 420 nm.

The stability of the milled raloxifene hydrochloride was measured over a seven day period under various temperature conditions. The results of the stability test are show below in Table 32.

TABLE 32

| Storage | | Mean/ | D50/ | D90/ | D95/ |
|---|---|---|---|---|---|
| condition time | Storage Condition | nm | nm | nm | nm |
| Time = 0 Days | Ambient | 195 | 187 | 254 | 283 |
| Time = 0 Days | Ambient | 213 | 190 | 375 | 420 |
| Time = 2 Days | 5° C. | 209 | 202 | 271 | 299 |
| Time = 2 Days | 5° C. | 220 | 211 | 287 | 320 |
| Time = 2 Days | 25° C. | 207 | 197 | 271 | 305 |
| Time = 2 Days | 25° C. | 206 | 193 | 279 | 323 |
| Time = 2 Days | 40° C. | 211 | 204 | 274 | 301 |
| Time = 2 Days | 40° C. | 210 | 202 | 276 | 305 |

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

We claim:

1. A stable nanoparticulate benzothiophene composition comprising:
(a) particles of a benzothiophene or a salt thereof having an effective average particle size of less than about 2000 nm; and
(b) at least one surface stabilizer.

2. The composition of claim 1, wherein the benzothiophene is raloxifene hydrochloride.

3. The composition of claim 1, wherein the benzothiophene is selected from the group consisting of a crystalline phase of benzothiophene, an amorphous phase of benzothiophene, a semi-crystalline phase of benzothiophene, a semi-amorphous phase of benzothiophene, and mixtures thereof.

4. The composition of claim 1, wherein the effective average particle size of the nanoparticulate benzothiophene particles is selected from the group consisting of less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 100 nm, less than about 75 nm, and less than about 50 nm.

5. The composition of claim 4, wherein the benzothiophene is raloxifene hydrochloride.

6. The composition of claim 5, wherein the composition is formulated:
(a) for oral, pulmonary, rectal, opthalmic, colonic, parenteral, intracisternal, intravaginal, intraperitoneal, local, buccal, nasal, or topical administration;
(b) into a dosage form selected from the group consisting of liquid dispersions, gels, aerosols, ointments, creams, controlled release formulations, fast melt formulations, lyophilized formulations, tablets, capsules, delayed release formulations, extended release formulations, pulsatile release formulations, and mixed immediate release and controlled release formulations; or
(c) a combination of (a) and (b).

7. The composition of claim 6, wherein the composition further comprises one or more pharmaceutically acceptable excipients, carriers, or a combination thereof.

8. The composition of claim 7, wherein:
(a) the benzothiophene is present in an amount selected from the group consisting of from about 99.5% to about 0.001%, from about 95% to about 0.1%, and from about 90% to about 0.5%, by weight, based on the total combined weight of the benzothiophene and at least one surface stabilizer, not including other excipients;
(b) at least one surface stabilizer is present in an amount selected from the group consisting of from about 0.5% to about 99.999% by weight, from about 5.0% to about 99.9% by weight, and from about 10% to about 99.5% by weight, based on the total combined dry weight of the benzothiophene and at least one surface stabilizer, not including other excipients; or
(c) a combination of (a) and (b).

9. The composition of claim 1, wherein the surface stabilizer is selected from the group consisting of a non-ionic surface stabilizer, an anionic surface stabilizer, a cationic surface stabilizer, a zwitterionic surface stabilizer, and an ionic surface stabilizer.

10. The composition of claim 9, wherein the benzothiophene is raloxifene hydrochloride.

11. The composition of claim 9, wherein the at least one surface stabilizer is selected from the group consisting of cetyl pyridinium chloride, gelatin, casein, phosphatides, dextran, glycerol, gum acacia, cholesterol, tragacanth, stearic acid, benzalkonium chloride, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, dodecyl trimethyl ammonium bromide, polyoxyethylene stearates, colloidal silicon dioxide, phosphates, sodium dodecylsulfate, carboxymethylcellulose calcium, hydroxypropyl celluloses, hypromellose, carboxymethylcellulose sodium, methylcellulose, hydroxyethylcellulose, hypromellose phthalate, noncrystalline cellulose, magnesium aluminum silicate, triethanolamine, polyvinyl alcohol, polyvinylpyrrolidone, 4-(1,1,3,3-tetramethylbutyl)-phenol polymer with ethylene oxide and formaldehyde, poloxamers; poloxamines, a charged phospholipid, dioctylsulfosuccinate, dialkylesters of sodium sulfosuccinic acid, sodium lauryl sulfate, alkyl aryl polyether sulfonates, mixtures of sucrose stearate and sucrose distearate, p-isononylphenoxypoly-(glycidol), decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; lysozyme, PEG-phospholipid, PEG-cholesterol, PEG-cholesterol derivative, PEG-vitamin A, PEG-vitamin E, random copolymers of vinyl acetate and vinyl pyrrolidone, a cationic polymer, a cationic biopolymer, a cationic polysaccharide, a cationic cellulosic, a cationic alginate, a cationic nonpolymeric compound, a cationic phospholipid, cationic lipids, polymethylmethacrylate trimethylammonium bromide, sulfonium compounds, polyvinylpyrrolidone-2-dimethylaminoethyl methacrylate dimethyl sulfate, hexadecyltrimethyl ammonium bromide, phosphonium compounds, quarternary ammonium compounds, benzyl-di(2-chloroethyl)ethylammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C₁₂₋₁₅-dimethyl hydroxyethyl ammonium chloride, C₁₂₋₁₅dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy)₄ ammonium chloride, lauryl dimethyl (ethenoxy)₄ ammonium bromide, N-alkyl (C₁₂₋₁₈)dimethylbenzyl ammonium chloride, N-alkyl (C₁₄₋₁₈)dimethyl-benzyl ammonium chloride, N-tetradecylidmethylbenzyl ammonium chloride monohydrate, dimethyl didecyl ammonium chloride, N-alkyl and (C₁₂₋₁₄) dimethyl 1-napthylmethyl ammonium chloride, trimethylammonium halide, alkyl-trimethylammonium salts, dialkyl-dimethylammonium salts, lauryl trimethyl ammonium chloride, ethoxylated alkyamidoalkyldialkylammonium salt, an ethoxylated trialkyl ammonium salt, dialkylbenzene dialkylammonium chloride, N-didecyldimethyl ammonium chloride, N-tetradecyldimethylbenzyl ammonium, chloride monohydrate, N-alkyl(C₁₂₋₁₄) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C₁₂ trimethyl ammonium bromides, C₁₅ trimethyl ammonium bromides, C₁₇ trimethyl ammonium bromides, dodecylbenzyl triethyl ammonium chloride, poly-diallyldimethylammonium chloride (DADMAC), dimethyl ammonium chlorides, alkyldimethylammonium halogenides, tricetyl methyl ammonium chloride, decyltrimethylammonium bromide, dodecyltriethylammonium bromide, tetradecyltrimethylammonium bromide, methyl trioctylammonium chloride, POLYQUAT 10™, tetrabutylammonium bromide, benzyl trimethylammonium bromide, choline esters, benzalkonium chloride, stearalkonium chloride compounds, cetyl pyridinium bromide, cetyl pyridinium chloride, halide salts of quaternized polyoxyethylalkylamines, MIRAPOL™, ALKAQUA™, alkyl pyridinium salts; amines, amine salts, amine oxides, imide azolinium salts, protonated quaternary acrylamides, methylated quaternary polymers, and cationic guar.

12. The composition of claim 11, wherein the benzothiophene is raloxifene hydrochloride.

13. The composition of claim 1, wherein:
(a) the AUC of the benzothiophene, when assayed in the plasma of a mammalian subject following administration, is greater than the AUC for a non-nanoparticulate benzothiophene formulation, administered at the same dosage;
(b) the Cmax of the benzothiophene, when assayed in the plasma of a mammalian subject following administration, is greater than the Cmax for a non-nanoparticulate benzothiophene formulation, administered at the same dosage;
(c) the Tmax of the benzothiophene, when assayed in the plasma of a mammalian subject following administration, is less than the Tmax for a non-nanoparticulate benzothiophene formulation, administered at the same dosage; or
(d) any combination of (a), (b), and (c).

14. The composition of claim 13, wherein the benzothiophene is raloxifene hydrochloride.

15. The composition of claim 1, additionally comprising one or more non-benzothiophene active agents.

16. The composition of claim 15, additionally comprising one or more active agents useful in treating osteoporosis, breast cancer, or a combination thereof.

17. The composition of claim 16, wherein the benzothiophene is raloxifene hydrochloride.

18. The composition of claim 16, additionally comprising at least one active agent selected from the group consisting of calcium supplements, vitamin D, bisphosphonates, bone formation agents, estrogens, parathyroid hormone, parathyroid hormone derivatives, selective receptor modulators, anticancer agents, and chemotherapy regimens.

19. The composition of claim 18, additionally comprising at least one active agent selected from the group consisting of risedronate sodium, ibandronate sodium, etidronate Disodium, teriparatide, alendronate, calcitonin, paclitaxel, doxorubicin, pamidronate disodium, anastrozole, exemestane, cyclophosphamide, epirubicin, toremifene, letrozole, trastuzumab, megestrol, Nolvadex, docetaxel, capecitabine, goserelin acetate, and zoledronic acid.

20. A method of making a nanoparticulate benzothiophene composition comprising:
contacting particles of s benzothiophene or a salt thereof with at least one surface stabilizer for a time and under conditions sufficient to provide a benzothiophene composition having an effective average particle size of less than about 2 microns.

21. The method of claim 20, wherein the contacting comprises grinding, wet grinding, homogenizing, or a combination thereof.

22. The method of claim 21, wherein the benzothiophene is raloxifene hydrochloride.

23. A method for the treatment or prevention of osteoporosis comprising administering to a subject in need an effective amount of a composition comprising:
(a) benzothiophene nanoparticles having an effective average particle size of less than about 2 microns;
(b) at least one surface stabilizer; and
(c) at least one pharmaceutically acceptable carrier.

24. The method of claim 23, wherein the benzothiophene is raloxifene hydrochloride.

25. A method for the treatment of breast cancer and other tumors of the breast and lymph nodular tissues comprising administering to a subject in need an effective amount of a composition comprising:
(a) benzothiophene nanoparticles having an effective average particle size of less than about 2 microns;
(b) at least one surface stabilizer; and
(c) at least one pharmaceutically acceptable carrier.

26. The method of claim 25, wherein the benzothiophene is raloxifene hydrochloride.
